(19) United States
(12) Patent Application Publication
(10) Pub. No.: US 2005/0152911 A1
(43) Pub. Date: Jul. 14, 2005

(54) Norovirus monoclonal antibodies and peptides

(75) Inventor: Michele Hardy, Bozeman, MT (US)

Correspondence Address: Robin M. Silva;Dorsey & Whitney LLP, Intellectual Property Department, Four Embarcadero Center, Suite 3400, San Francisco, CA, 94111-4187, US

(73) Assignee: Montana State University, Bozeman, MT (US)

(21) Appl. No.: 10/950,163

(22) Filed: Sep. 24, 2004

Related U.S. Application Data
(60) Provisional application No. 60/508,262, filed on Sep. 24, 2003.

(51) Int. Cl. A61K 39/42; C07K 14/01; C07K 16/08

(52) U.S. Cl. 424/159.1; 530/350; 530/388.3

(57) ABSTRACT

The present invention is drawn to monoclonal antibodies that bind to a Norovirus, peptides that inhibit monoclonal antibody binding to a Norovirus, and peptides that inhibit binding of a Norovirus to a cell. The compositions of the invention find use as Norovirus immunogens, therapeutics, diagnostics, and vaccines.

### 2. PRIORITY AND RELATED APPLICATIONS

This application claims the benefit for the filing date of U.S. Provisional Patent Application Ser. No. 60/508,262, filed Sep. 24, 2003, pending. All priority and related applications are hereby incorporated by reference in their entirety.

### 1. STATEMENT AS TO FEDERALLY SPONSORED RESEARCH

This invention was made with government support under Grant Number DAMD 17-01-C-0040 awarded by the United States Army/MRMC and Grant Number AI-43450 awarded by the National Institutes of Health.

### 3. BACKGROUND

The Norovirus genus of the family Caliciviridae comprises morphologically similar but antigenically diverse viruses that are the most common cause of nonbacterial epidemics of acute gasteroenteritis. (Huang et al., J. I. D. 188:19-31 (2003)) Noroviruses are transmitted primarily by consumption of contaminated food or water; however, direct transmission from person-to-person may occur. Symptoms of Norovirus infection include nausea, vomiting, watery, non-bloody diarrhea, abdominal cramps, headache, fever, chills, myalgias, and sore throat. Fluid loss causes dehydration, which is the most common complication of Norovirus disease. Symptoms usually last from 24 to 60 hours and recovery is usually complete with no serious, long term sequelae.

The nucleotide and deduced amino acid sequences of Norovirus genomic RNA are available for a number of isolates. (see, e.g., Dingle et al. J. Gen. Virol. 76(Pt9):2349-2355 (1999); Green et al. J. Infect. Dis. 185:133-146 (2000); Hale et al. Clinical and Diagnostic Laboratory Immunology 6:142-145 (1999); Jiang et al. Virology 195:51-61 (1993); Jiang et al. J. Med. Virol. 47:309-316 (1995); King et al. Virus Genes 15:5-7 (1997); Kobayashi et al. J. Clin. Microbiol. 38:3492-3494 (2000); Lambden et al., Science 259:516-519 (1993); Lambden et al. Virus Genes 10:149-152 (1995); Lew et al. Virology 200:319-325 (1994); Liu et al. Arch. Virol. 1140:1345-1356 (1995); Someya et al. Virology 278:490-500 (2000)). Despite the advances in Norovirus molecular biology, the mechanisms of immunity, virus-cell interactions, and potential targets of antiviral therapies have not been elucidated. These studies have been hindered because a tissue culture system and animal model for Noroviruses are not available. Therefore, humoral and cellular immune responses to Noroviruses and the role of viral gene products in pathogenicity have not been rigorously examined. Volunteer studies have established a role for antibody in resistance to Norovirus challenge but the immunity is short-lived and strain specific. Long term immunity lasting about 27 to 42 months has been observed in challenge studies but long term immunity does not correlate with pre-challenge serum antibody titers or the develop of an antiviral antibody response.

Therefore, there is a need for methods to determine the regions of Norovirus proteins that are targets of protective immunity and that interact with host cells. The identification of these regions provides the basis for Norovirus diagnostic reagents, therapies, and vaccines.

### 3. SUMMARY

This invention relates generally to monoclonal antibodies (MAbs) that bind the capsid protein of a Norovirus and the identification of regions of the capsid protein that are recognized by the MAbs. More specifically, the invention provides methods of identifying Norovirus capsid protein epitopes and determining the amino acid composition of capsid protein epitopes. The compositions of the invention find use as immunogens, vaccines, antiviral therapeutic agents, and diagnostic reagents.

In one embodiment, the present invention provides antibodies that bind to a Norovirus capsid protein. In a preferred embodiment, the antibody competes with a second antibody for binding a Norovirus capsid protein.

In another embodiment, the present invention provides a peptide that blocks the binding of a Norovirus capsid antibody to a Norovirus.

In another embodiment, the present invention provides peptides that block the binding of a Norovirus to a cell.

In some embodiments, an MS peptide can have the general formula or sequence X₅₀—X₅₁—X₅₂—X₅₃—X₅₄—X₅₅—X₅₆—X₅₇—X₅₈ (SEQ ID NO:142), wherein X₅₀ is selected from the group consisting of W and P, with W being preferred; X₅₁ selected from the group consisting of S, I, T, G, H and N, with T being preferred; X₅₂ is selected from the group consisting of R, L, F and I, with R being preferred; X₅₃ is selected from the group consisting of G, Q, S, D, P, T, A and K, with G being preferred; X₅₄ is any amino acid, with preferred amino acids selected from the group consisting of Q, G, M, E, W, S, L, T, I, A, V and N; X₅₅ is selected from the group consisting of E, D, R, Q, H and P, with H being preferred; X₅₆ is any amino acid, with preferred amino acids selected from the group consisting of R, F, Q, N, T, G, K and S; X₅₇ is selected from the group consisting of L, I, D and V, with L being preferred; and X₅₈ is any amino acid, with preferred amino acids selected from the group consisting of S, K, A, Q, V, Y, H, L, T and W.

In some embodiments, an MS peptide comprises the sequence W-T-R-G-X₅₄—H—X₅₆-L-X₅₈ (SEQ ID NO:143).

In some embodiments, an MS peptide comprises the sequence X₅₀—X₅₁—X₅₂—X₅₃—X₅₄—X₅₅—X₅₆—X₅₇—X₅₈ (SEQ ID NO:142), wherein X₅₀ is selected from the group consisting of W and P, with W being preferred; X₅ selected from the group consisting of S, I, T and N, with T being preferred; X₅₂is selected from the group consisting of R, L and I, with R being preferred; X₅₃ is selected from the group consisting of G, Q, S, D and K, with G being preferred; X₅₄ is any amino acid, with preferred amino acids selected from the group consisting of Q, G, M, E, W, S and N; X₅₅ is selected from the group consisting of H and P, with H being preferred; X₅₆ is any amino acid, with preferred amino acids selected from the group consisting of R, F, Q, N, T, K and S; X₅₇ is selected from the group consisting of L, I, D and V, with L being preferred; and X₅₈ is any amino acid, with preferred amino acids selected from the group consisting of S, K, A, Q, V, Y, H, L and W.

In some embodiments, an MS peptide comprises the sequence W-T-X₅₂—X₅₃—X₅₄—X₅₅—X₅₆-L-X₅₈ (SEQ ID NO:144), wherein X₅₂ is selected from the group consisting of R and F; X₅₃ is selected from the group consisting of P and G; X₅₄ is selected from the group consisting of S, G and Q; X₅5 is selected from the group consisting of H and E; X₅₆ is selected from the group consisting of N, G and Q; and X₅₈ is selected from the group consisting of S and T.

In some embodiments, MS peptides exemplified by X₅₀—X₅₁—X₅₂—X₅₃—X₅₄—X₅₅—X₅₆-X₅₇—X₅₈ (SEQ ID NO:142), W-T-R-G-X₅₄—H—X₅₆-L-X₅₈ (SEQ ID NO:143), and W-T-X₅₂—X₅₃—X₅₄—X₅₅—X₅₆-L-X₅₈ (SEQ ID NO:144) can, in some embodiments, further comprise (i) one or more amino acids at the amino-terminus, and/or (ii) further comprise one or more amino acids at the carboxy-terminus. In some embodiments, one or more of the amino acids of MS peptides exemplified by X₅₀—X₅₁—X₅₂—X₅₃—X₅₄—X₅₅—X₅₆—X₅₇—X₅₈ (SEQ ID NO:142), W-T-R-G-X₅₄—H—X₅₆-L-X₅₈ (SEQ ID NO: 143), and W-T-X₅₂—X₅₃—X₅₄—X₅₅—X₅₆-L-X₅₈ (SEQ ID NO:144) can, in some embodiments, correspond to Norovirus sequences as depicted in FIG. 8.

In some embodiments, an MS peptide can comprise the formula or sequence X₆₀—X₆₁—P-A-P—X₆₂—X₆₃—X₆₄—X₆₅ (SEQ ID NO:145), wherein X₆₀ is selected from the group consisting of W, D, E, G, S and A, with W being preferred; X₆₁ selected from the group consisting of L, I, V and a deletion at the position, with L being preferred; X₆₂ is selected from the group consisting of I, L, V and A, with I and L being preferred, and I being particularly preferred; X₆₃ is selected from the group consisting of D and G, with D being preferred; X₆₄ is selected from the group consisting of K, V, T and F, with K and F being preferred, and K being particularly preferred; and X₆₅ is selected from the group consisting of L and P.

In some embodiments, an MS peptide as exemplified by X₆₀—X₆₁—P-A-P—X₆₂—X₆₃—X₆₄—X₆₅ (SEQ ID NO:145), can, in some embodiments, further comprise (i) one or more amino acids at the amino-terminus, and/or (ii) one or more amino acids at the carboxy-terminus. In some embodiments, an MS peptide as exemplified by X₆₀—X₆₁—P-A-P—X₆₂—X₆₃—X₆₄—X₆₅ (SEQ ID NO:145), can comprise one or more amino acids that corresponds to a Norovirus sequences as depicted in FIG. 9.

In a preferred embodiment, the peptides of the present invention comprise amino acids sequences WTRGSHNL (SEQ ID NO:1), WTRGGHGL, (SEQ ID NO:2), WTRGQHQL (SEQ ID NO:3), or WLPAPIDKL (SEQ ID NO:4).

In other aspects, the invention provides methods of blocking binding of an antibody to a Norovirus. In one embodiment, binding of a labeled antibody to a Norovirus can be blocked by the binding of another, preferably unlabeled antibody to the virus. In another embodiment, antibody binding can be inhibited by binding the antibody to a peptide of the invention.

In other aspects, the present invention provides antibodies that bind to the capsid protein of a Norovirus, and prevent adhesion or binding of the virus to a cell. In some embodiments, preventing binding of the virus to a cell thereby prevents infection of the cell. In a preferred embodiment the antibody is NV54.6, NV72.10, or SMV61.21. Accordingly, in other aspects, the present invention provides antibodies and methods of use as both a therapeutic or preventative treatment of a Norovirus.

In another aspect, the present invention provides peptides that inhibit binding of a Norovirus to a cell. In some embodiments, preventing binding of the virus to a cell thereby prevents infection of the cell. In a preferred embodiment a peptide of the invention is 1730 (SEQ ID NO:1), 1731 (SEQ ID NO:2), 1732 (SEQ ID NO:3), or 1800 (SEQ ID NO:4). Accordingly, in other aspects, the present invention provides peptides and methods of use as both a therapeutic or preventative treatment of NV.

In another aspect, the present invention provides peptides that induce an immune response in a host, thus preventing infection or lessening NV disease. In a preferred embodiment a peptide of the invention is 11730 (SEQ ID NO:1), 1731 (SEQ ID NO:2), 1732 (SEQ ID NO:3), or 1800 (SEQ ID NO:4). Accordingly, in other aspects, the present invention provides peptides and methods of use as both a therapeutic or preventative vaccine for a Norovirus.

In another aspect, the present invention provides antibodies and peptides that find use as diagnostic agents.

### 4. BRIEF DESCRIPTION OF THE DRAWINGS

FIG. 1 is a diagram of the prototype Norovirus genomic RNA, Norwalk virus (NV) which contains three major open reading frames (ORF1, -2, -3) and a polyadenylated 3′-end. ORF1 encodes a polyprotein which is cleaved to yield NTPase, VPg, viral protease (Pro), and RNA-dependent RNA polymerase (RdRp). Open triangles and letters indicate the cleavage sites in the polyprotein processed by Pro. ORF2 encodes the capsid protein (VP1) which is cleaved at K²²⁷/T; however, cleaved capsid protein is not detected in the virion. ORF3 encodes a minor capsid protein (VP2). VPg is covalently attached to the 5′-terminus of genomic RNA and a subgenomic mRNA transcribed from ORF2.

FIG. 2 is an electron micrograph of recombinant Norwalk virus-like particles (rNV, VLPs), a 3D cryo-reconstruction of rNV VLPs, and an X-ray crystal structure of NV capsid protein. “A”, “B”, and “C” are quasi-equivalent subunits that constitute a T=3 lattice. “3” and “5” indicate the locations of the 5- and 3-fold axes. “N” is the amino-terminal arm (amino acids 1049), which faces the interior of the capsid. “S” is the shell domain (amino acids 50-225). “P” is the protruding arm domain that forms capsomeres that extend from the virion surface. P domain is divided into two subdomains, P1 (amino acids 226-278 and 406-520) and P2 (amino acids 279-405). S and P domains are connected by a flexible hinge region. (Prasad et al., Science 286:287-290 (1999))

FIG. 3 is a bar graph of the results of blocking experiments in which MAb NV54.6 inhibits binding of rNV·VLP to differentiated CaCo-2 cells in a dose dependent manner.

FIG. 4 is a bar graph of the results of blocking experiments in which peptides 1730 (WTRGSHNL: SEQ ID NO:1), 1731 (WTRGGHGL: SEQ ID NO:2), and 1732 (WTRGQHQL: SEQ ID NO:3) inhibit binding of MAb NV54.6 to rNV VLP. IRR 1794 (RVYIHPF: SEQ ID NO:5) is from human angiotensin III. (see U.S. Pat. No.5,854,388)

FIG. 5 show the results of Norovirus hemagglutination and Norovirus hemagglutination inhibition. Panel A shows rNV VLP hemagglutination at the indicated VLP concentration. Panel B shows non-agglutinated RBCs in PBS. Panel C shows hemagglutination of murine-adapted influenza virus. Panel D shows the inhibition of hemagglution of rNV VLPs by MAb NV54.6. Panel E shows that NV54.6 does not inhibit hemagglutination by influenza virus. Panel F shows the inhibition of hemagglutination of rNV VLPs by MAb NV72.10. Panel G shows that NV72.10 does not inhibit hemagglutination by influenza virus. Panel H shows that MAb DREG55 (negative control) does not inhibit hemagglutination of rNV VLP. Panel I shows that MAb DREG55 does not hemagglutinate. Panel J shows that MAb El86.10 does not inhibit hemagglutination of rNV VLPs. Panel K shows that MAb SMV61.21 inhibits hemagglutination of rSMV VLPs. Panel L shows hemagglutination by rSMV VLPs. Panel M shows non-agglutinated RBCs in PBS.

FIG. 6 shows the reactivity of SMV61.21 with boiled and/or β-mercaptoethanol treated SMV capsid proteins in a Western blot.

FIG. 7 shows the reactivity of NV54.6, NV72.10, and SMV61.21 with the indicated Norovirus VLPs in a non-denaturing dot blot.

FIG. 8 shows a partial sequence of Norwalk virus capsid protein and sequences corresponding thereto from the indicated Noroviruses. Chia virus (SEQ ID NO:6), Desert Shield virus (SEQ ID NO:7), Grimsby virus (SEQ ID NO:8), Hawaii virus (SEQ ID NO:9), Lordsdale virus (SEQ ID NO:10), Maryland virus/145 (SEQ ID NO:1 1), Mexico virus (SEQ ID NO:12), Norwalk virus (SEQ ID NO:13), Seto virus (SEQ ID NO: 14), Snow Mountain virus (SEQ ID NO:15), Southampton virus (SEQ ID NO:16).

FIG. 9 shows a particle sequence of Snow Mountain virus capsid protein and sequences corresponding thereto from the indicated Noroviruses. Chia virus (SEQ ID NO:17), Desert Shield virus (SEQ ID NO:18), Grimsby virus (SEQ ID NO:19), Hawaii virus (SEQ ID NO:20), Lordsdale virus (SEQ ID NO:21), Maryland virus/145 (SEQ ID NO:22), Mexico virus (SEQ ID NO:23), Norwalk virus (SEQ ID NO:24), Seto virus (SEQ ID NO:25), Snow Mountain virus (SEQ ID NO:26), Southampton virus (SEQ ID NO:27).

FIG. 10 shows the amino acid sequence of a NV capsid protein (SEQ ID NO:28). (Jiang et al. Virology 195(1):51-61 (1993))

FIG. 11 shows the amino acid sequence of a NV capsid protein (SEQ ID NO:29). (Kobayashi et al. J. Clin. Microbiol. 38(9):3492-3494 (2003))

FIG. 12 shows the amino acid sequence of a SMV capsid protein (SEQ ID NO:30). (Lochridge et al. Virus Genes 26:71-82 (2003))

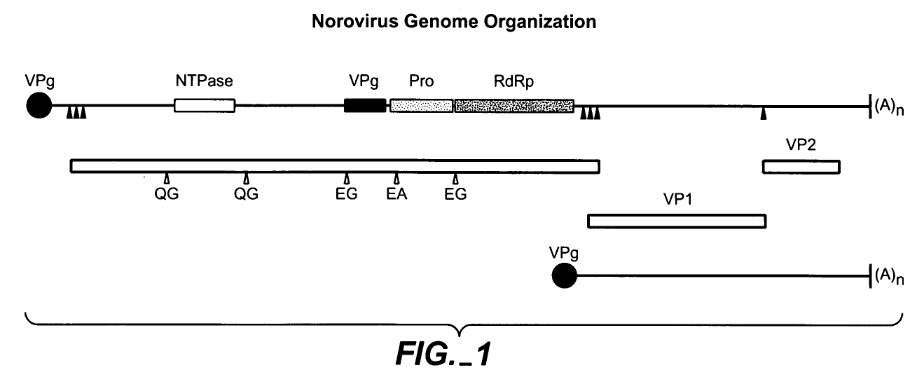

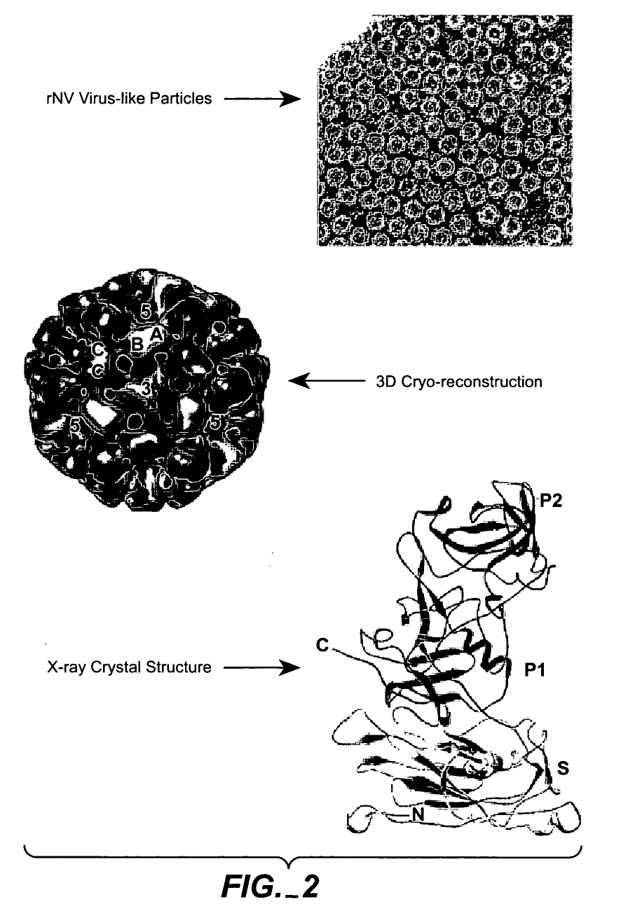

### 5. DETAILED DESCRIPTION

The present invention is directed to the discovery that certain antibodies can bind to the capsid protein of a Norovirus and can prevent adhesion or binding of a Norovirus to a cell. These antibodies, herein termed “MS antibodies”, include, for example, monoclonal antibodies (MAbs), such as, NV54.6, NV72.10, and SMV61.21. Thus, MS antibodies that can bind the same, related, or corresponding epitope, as NV54.6, NV72.10, and/or SMV61.21 find use as both a therapeutic or preventative treatment of Norovirus infection or disease. MS antibodies have been shown to bind several peptides (herein termed “MS peptides”), identified through a phage display screen. The MS peptides can be utilized as an immunogen, e.g. as a therapeutic composition, including but not limited to, a vaccine, to produce an immune response that can prevent, ameliorate, or treat Norovirus infection, or as a therapeutic peptide that can compete for Norovirus binding to a cell.

Thus, in addition to compositions, the present invention provides methods of inhibiting adhesion of a Norovirus to a cell. In some embodiments, reducing or eliminating Norovirus binding to a cell may decrease infectivity. In some embodiments, this includes methods of inhibiting adhesion of a Norovirus to a host cell by MS antibodies or MS peptides of the invention. The methods find use in the treatment of Norovirus disease, the identification of a Norovirus, and the diagnosis of a Norovirus disease in a host or patient.

In some embodiments, the methods comprise inhibiting the interaction of the binding of the capsid protein and the corresponding ligand on a cell. The site of interaction is the epitope to which an MS antibody binds, which may correspond to the capsid protein region bound by the cell. In some embodiments, the epitope to which an MS antibody binds may be adjacent according to the primary or tertiary structure of the capsid protein site of interaction with a cell ligand, accordingly by sterically inhibit such interaction. Thus, binding of an MS antibody to a Norovirus can inhibit directly or indirectly (e.g., sterically) the interaction of the capsid protein with a cell ligand. In some embodiments, a cell is a host cell and the binding of a MS antibody can inhibit infection of the host cell by a Norovirus.

In some embodiments, the methods comprise inhibiting the interaction of the binding of the capsid protein and the corresponding ligand on a cell. In some embodiments, a peptide of the invention can be bound to the corresponding ligand on a cell. The binding of the peptide to the ligand inhibits Norovirus binding to the cell. In some embodiments, a cell is a host cell and the binding of a peptide thereto can inhibit infection of the host cell by a Norovirus.

Accordingly, the present invention provides antibodies that bind to a Norovirus and compete with an MS antibody. By “Norovirus”, “Norovirus (NOR)”, “norovirus” and grammatical equivalents herein are meant members of the genus Norovirus of the family Caliciviridae. In some embodiments, a Norovirus can include a group of related, positive-sense single-stranded RNA, nonenveloped viruses that can be infectious to human or non-human mammalian species. In some embodiments, a Norovirus can cause acute gastroenteritis in humans. Noroviruses also can be referred to as small round structured viruses (SRSVs) having a defined surface structure or ragged edge when viewed by electron microscopy. Included within the Noroviruses are at least four genogroups (GI-IV) defined by nucleic acid and amino acid sequences, which comprise 15 genetic clusters. The major genogroups are GI and GII. GIII and GIV are proposed but generally accepted. Representative of GIII is the bovine, Jena strain. GIV contains one virus, Alphatron, at this time. For a further description of Noroviruses see Vinje et al. J. Clin. Micro. 41:1423-1433 (2003). By “Norovirus ” also herein is meant recombinant Norovirus virus-like particles (rNOR VLPs). In some embodiments, recombinant expression of at least the Norovirus capsid protein encoded by ORF2 in cells, e.g., from a baculovirus vector in Sf9 cells, can result in spontaneous self-assembly of the capsid protein into VLPs. In some embodiments, recombinant expression of at least the Norovirus proteins encoded by ORF1 and ORF2 in cells, e.g., from a baculovirus vector in Sf9 cells, can result in spontaneous self-assembly of the capsid protein into VLPs. VLPs are structurally similar to Noroviruses but lack the viral RNA genome and therefore are not infectious. Accordingly, “Norovirus” includes virions that can be infectious or non-infectious particles, which include defective and defective-interfering particles.

Non-limiting examples of Noroviruses include Norwalk virus (NV, GenBank M87661, NP_{—}056821), Southampton virus (SHV, GenBank L07418), Desert Shield virus (DSV, U04469), Hesse virus (HSV), Chiba virus (CHV, GenBank AB042808), Hawaii virus (HV, GenBank U0761 1), Snow Mountain virus (SMV, GenBank U70059), Toronto virus (TV, Leite et al., Arch. Virol. 141:865-875), Bristol virus (BV), Jena virus (JV, AJ01099), Maryland virus (MV, AY032605), Seto virus (SV, GenBank AB031013), Camberwell (CV, AF145896), Lordsdale virus (LV, GenBank X86557), Grimsby virus (GRV, Hale et al., Clinical and Diagnostic Laboratory Immunology 6:142-145), Mexico virus (MXV, GenBank U22498). The nucleic acid and corresponding amino acid sequences of each are all incorporated by reference in their entirety. In some embodiments, a cryptogram can be used for identification purposes and is organized: host species from which the virus was isolated/genus abbreviation/species abbreviation/strain name/year of occurrence/country of origin. (Green et al., Human Caliciviruses, in Fields Virology Vol. 1 841-874 (Knipe and Howley, editors-in-chief, 4th ed., Lippincott Williams & Wilkins 2001)). Norwalk virus and Snow Mountain virus are preferred in some embodiments.

The present invention provides a variety of proteins including Norovirus proteins (including capsid proteins) and MS peptides. By “protein” herein is meant at least two covalently attached amino acids, which includes proteins, polypeptides, oligopeptides and peptides. In some embodiments, the at least two covalently attached amino acids are attached by a peptide bond. The protein may be made up of naturally occurring amino acids and peptide bonds, or synthetic peptidomimetic structures, i.e. “analogs”, such as peptoids (see Simon et al., PNAS USA 89(20):9367 (1992)), which can be resistant to proteases or other physiological and/or storage conditions. Thus, peptidomimetic structures can be preferred when MS peptides are to be administered to a patient. Thus “amino acid” or “peptide residue” as used herein means both naturally occurring and synthetic amino acids, which contain an amino group, a carboxyl group, a hydrogen atom, and an R-group or “side chain” bonded to a carbon atom. Therefore, in some embodiments, homophenylalanine, citrulline, omithine, and norleucine can be considered amino acids for the purposes of this disclosure. “Amino acid” also includes imino acid residues such as proline and hydroxyproline. The amino acid “R group” or “side chain” may be in either the (R) or the (S) configuration. In a preferred embodiment, the amino acids are in the (S) or L-configuration. In various exemplary embodiments, an amino acid side chain can have an aromatic (e.g., phenylalanine, tyrosine, tryptophan), a sulfur (e.g., cysteine, cystine, methione), an hydroxyl (e.g., serine, threonine, tyrosine), a basic (e.g., lysine (—NH₃), arginine (guanidinium), histidine (imidazole)), an acidic (asparatate (—COOH), glutamate (—COOH), asparagine (—CONH₂), glutamine (—CONH₂)), an alphatic (e.g., glycine, alanine, valine, leucine, isoleucine), and/or an alkyl having from about 1 to 5 linear or branched saturated carbon chain (e.g., alanine, valine, leucine, isoleucine) group. In some embodiments, an amino acid side chain can be attached to the α, β, and/or γcarbon etc. (Stryer. Biochemistry 1542 (3d ed. W.H. Freeman & Co. 1988)). If non-naturally occurring side chains are used, non-amino acid substituents may be used, for example to prevent or retard in vivo degradation. By “naturally occurring amino acid” and grammatical equivalents herein are meant an amino acid that can be produced by a cell as it is found in nature. In various exemplary embodiments, a naturally occurring amino acid can be glycine (G), alanine (A), valine (V), leucine (L), isoleucine (1), proline (P), phenylalanine (F), tyrosine (T), tryptophan (W), cysteine (C), methionine (M), serine (S), threonine (T), lysine (K), arginine (A), histidine (H), aspartate (D), glutamate (E), aspargine (N), glutamine (Q), hydroxyproline.

By “Norovirus peptide”, “NOR peptide”, and grammatical equivalents herein are meant a protein comprising a sequence homologous or identical to an amino acid sequence deduced from a Norovirus ORF. In some embodiments, a NOR peptide is about 5 to about 150 amino acids in length. In a preferred embodiment, an NOR peptide is about 5 to about 30 amino acids in length. In an even more preferred embodiment, an NOR peptide is about 5 to about 15 amino acids in length. In an even more preferred embodiment, a NOR peptide is about 8 to about 20 amino acids with peptides of 7, 8, 9, 10,11, 12, 13, 14, 15, 16, 17, 18, and 19 all included. Thus, in some embodiments, a NOR peptide may be shorter than the sequence deduced from a Norovirus ORF. In some alternative embodiments, a NOR peptide can be longer than the amino acid sequence deduced from a Norovirus ORF as described below for fusion proteins and the like.

By “capsid peptide” and grammatical equivalents herein are meant a NOR peptide comprising a sequence homologous or identical to the deduced amino acid sequence of ORF2 of a Norovirus. In some embodiments, a peptide is a “capsid peptide” if it comprises a sequence of amino acids having homology to a sequence deduced from a Norovirus ORF2 as described herein. “Homology” in this context that is greater than about 75%, more preferably greater than about 80%, even more preferably greater than about 85% and most preferably greater than about 90%. In some embodiments the homology can be at least about 93 to 95 or 98%. The exact homology also can be determined based on the length of the peptide. Thus, a preferred homology for peptides from about 7 to about 15 residues in length can have about 1 or 2 amino acid substitutions, insertions, and/or deletions. In some embodiments, the sequence homologous or identical to the deduced amino acid sequence of a Norovirus ORF2 is about 5 to about 150 amino acids in length. In a preferred embodiment, the homologous or identical sequence is about 5 to about 30 amino acids in length. In an even more preferred embodiment, the homologous sequence is about 5 to about 15 amino acids in length. In an even more preferred embodiment, the homologous sequence is about 8 to about 20 amino acids in length. Homology in this context means sequence similarity or identity, with identity being preferred. This homology will be determined using standard techniques known in the art as described below.

In various exemplary embodiments, an MS peptide can be produced by organic synthesis techniques as known in the art or by recombinant techniques, e.g., through the expression of a recombinant nucleic acid as described below. A recombinant peptide can be distinguished from naturally occurring protein or peptide by at least one or more characteristics. For example, the peptide may be isolated or purified away from some or all of the matter and/or compounds with which it is normally associated in its wild type host, and thus may be substantially pure. For example, an isolated peptide can be unaccompanied by at least some of the material with which it is normally associated in its natural state, preferably constituting at least about 0.5%, more preferably at least about 5% by weight of the total matter in a given sample. A substantially pure peptide comprises at least about 75% by weight of the total protein, with at least about 80% being preferred, and at least about 90% being particularly preferred. In some embodiments, when expressed from a recombinant nucleic acid, the peptide may be made at a significantly higher concentration than is normally seen, through the use of a inducible promoter or high expression promoter, such that the peptide can be made at increased concentration levels. Alternatively, the peptide may be in a form not normally found in nature, including but not limited to, as in the addition of an epitope tag or amino acid substitutions, insertions and deletions, a fusion partner, as discussed below.

In a preferred embodiment, the invention provides MS peptides for use in a variety of applications, as outlined below. In some embodiments, an “MS peptide” refers to a capsid peptide, including but not limited to capsid fragments and synthetic peptides (e.g., peptide synthesized by organic chemical reactions or synthesized from a recombinant nucleic acid). In some embodiments, an MS peptide can be highly homologous to a capsid protein sequence, as described above. However, in alternative embodiments, an MS peptide may not be highly homologous to a capsid protein sequence. In some embodiments, an MS peptide can mimic either a conformational or linear epitope of a capsid protein. Thus, in various exemplary embodiments, an “MS peptide” can a) exhibit the ability to block binding of an MS antibody to a Norovirus ; b) exhibit the ability to block binding of a Norovirus to a cell; c) induce antibody cross-reactive with a Norovirus ; d) exhibit at least one biological activity of a naturally-occurring capsid protein; and/or e) have at least the indicated homology. In a preferred embodiment, an MS peptide can exhibit two or more of these characteristics. In a preferred embodiment, MS peptides can share at least one antigenic epitope with a naturally occurring protein (again, either a linear or conformational epitope), although in some embodiments this many not be required. In various exemplary embodiments, an MS peptide of the present invention may be shorter or longer than the naturally occurring, deduced amino acid sequences. In a preferred embodiment, an MS peptide can include portions or fragments of the sequences depicted herein. In a preferred embodiment an MS peptide can inhibit an antibody binding to a Norovirus and/or inhibit Norovirus binding to a cell.

In a preferred embodiment an “MS peptide” includes a peptide that induces an MS antibody that binds to an amino acid sequence deduced from a Norovirus ORF2. As known in the art, an antibody specifically binds to an epitope. (Berzofsky et al. Immunogenicity and Antigen Structure, in Fundamental Immunology 631-684 (Paul, ed. 5th ed., Lippincott Williams & Wilkins 2003)) By “epitope”, “antigenic determinant”, and grammatical equivalents herein are meant a region of an antigen or immunogen that can be specifically bound by a product of an immune response (e.g., antibody, immune cells), which includes residues that make contact with complementary residues in an antibody-combining site or T-cell receptor and or can induce an immune response. (Berzofsky et al. Immunogenicity and Antigen Structure, in Fundamental Immunology 637 (Paul, ed. 5th ed., Lippincott Williams & Wilkins 2003)) As the skilled artisan will appreciate, an epitope can be linear or conformational. “Linear epitope” refers to an epitope comprising a sequence of at least about 5 and not more than about 20 amino acids connected in a linear fashion, which amino acids, by themselves or as part of a larger sequence, bind to an antibody generated in response to such sequence. “Conformational epitope” refers to an epitope whose three dimensional, secondary and/or tertiary structure can be a substantial aspect of antibody binding. Generally but not uniformly, amino acids that comprise a conformational epitope do not comprise a linear sequence of a protein's primary structure. Thus, a conformational epitope may be shared by proteins having non-homologous linear amino acid sequences. Without being bound by theory, a conformational epitope can be shared because the tertiary structure recognized by an antibody can be shared between two or more amino acid sequences. Thus, in some embodiments, an MS peptide of the present invention can mimic the conformational structure of a naturally occurring Norovirus protein such that it binds antibody produced in response to the naturally occurring Norovirus protein and/or induces an antibody that binds to a naturally occurring Norovirus protein.

In some embodiments, MS peptides are functionally defined by their ability to compete for binding of an MS antibody. That is, MS antibodies such as NV54.6, NV72.10 and/or SMV62.21 bind to Norovirus at particular epitopes outlined herein, and peptides that compete for such binding are MS peptides.

In some embodiments, an MS peptide can have the general formula or sequence X₅₀—X₅₁—X₅₂—X₅₃—X₅₄—X₅₅—X₅₆—X₅₇—X₅₈ (SEQ ID NO:142), wherein X₅₀ is selected from the group consisting of W and P, with W being preferred; X₅₁ selected from the group consisting of S, I, T, G, H and N, with T being preferred; X₅₂ is selected from the group consisting of R, L, F and I, with R being preferred; X₅₃ is selected from the group consisting of G, Q, S, D, P, T, A and K, with G being preferred; X₅₄ is any amino acid, with preferred amino acids selected from the group consisting of Q, G, M, E, W, S, L, T, I, A, V and N; X₅₅ is selected from the group consisting of E, D, R, Q, H and P, with H being preferred; X₅₆ is any amino acid, with preferred amino acids selected from the group consisting of R, F, Q, N, T, G, K and S; X₅₇ is selected from the group consisting of L, I, D and V, with L being preferred; and X₅₈ is any amino acid, with preferred amino acids selected from the group consisting of S, K, A, Q, V, Y, H, L, T and W.

In some embodiments, an MS peptide comprises the sequence W-T-R-G-X₅₄—H—X₅₆-L-X₅₈ (SEQ ID NO:143).

In some embodiments, an MS peptide comprises the sequence X₅₀—X₅₁—X₅₂—X₅₃—X₅₄—X₅₅—X₅₆—X₅₇—X₅₈ (SEQ ID NO:142), wherein X₅₀ is selected from the group consisting of W and P, with W being preferred; X₅₁ selected from the group consisting of S, I, T and N, with T being preferred; X₅₂ is selected from the group consisting of R, L and I, with R being preferred; X₅₃ is selected from the group consisting of G, Q, S, D and K, with G being preferred; X₅₄ is any amino acid, with preferred amino acids selected from the group consisting of Q, G, M, E, W, S and N; X₅₅ is selected from the group consisting of H and P, with H being preferred; X₅₆ is any amino acid, with preferred amino acids selected from the group consisting of R, F, Q, N, T, K and S; X₅₇ is selected from the group consisting of L, I, D and V, with L being preferred; and X₅₈ is any amino acid, with preferred amino acids selected from the group consisting of S, K, A, Q, V,Y, H, Land W.

In some embodiments, an MS peptide comprises the sequence W-T-X₅₂—X₅₃—X₅₄—X₅₅—X₅₆-L-X₅₈ (SEQ ID NO:144), wherein X₅₂ is selected from the group consisting of R and F; X₅₃ is selected from the group consisting of P and G; X₅₄ is selected from the group consisting of S, G and Q; X₅₅ is selected from the group consisting of H and E; X₅₆ is selected from the group consisting of N, G and Q; and X₅₈ is selected from the group consisting of S and T.

In some embodiments, MS peptides exemplified by X₅₀—X₅₁—X₅₂—X₅₃—X₅₄—X₅₅—X₅₆—X₅₇—X₅₈ (SEQ ID NO:142), W-T-R-G-X₅₄—H—X₅₆-L-X₅₈ (SEQ ID NO:143), and W-T-X₅₂—X₅₃—X₅₄—X₅₅—X₅₆-L-X₅₈ (SEQ ID NO:144) can, in some embodiments, further comprise (i) one or more amino acids at the amino-terminus, and/or (ii) further comprise one or more amino acids at the carboxy-terminus. In some embodiments, one or more of the amino acids of MS peptides exemplified by X₅₀—X₅₁—X₅₂—X₅₃—X₅₄—X₅₅—X₅₆—X₅₇—X₅₈ (SEQ ID NO:142), W-T-R-G-X₅₄—H—X₅₆-L-X₅₈ (SEQ ID NO:143), and W-T-X₅₂—X₅₃—X₅₄—X₅₅—X₅₆-L-X₅₈ (SEQ ID NO:144) can, in some embodiments, correspond to Norovirus sequences as depicted in FIG. 8.

In some embodiments, an MS peptide can have the general sequence W—X₁—X₂—X₃—X₄—X₅—X₆—X₇—X₈ (SEQ ID NO:97), wherein X₁ can be I, N, S, or T; X₂ can be I, L, or R; X₃ can be D, G, K, Q, or S; X₄ can be D, E, G, N, M, Q, S, or W; X₅ can be H or P; X₆ can be F, K, N, Q, R, S, or T; X₇ can be D, I, L, or V; and X₈ can be A, H, K, L, Q, S, V, W, or Y.

In some embodiments, an MS peptide can have the general sequence W—X₁—X₂—X₃—X₄—X₅—X₆—X₇ (SEQ ID NO:98), wherein X₁ can be I, N, S, or T; X₂ can be I, L, or R; X₃ can be D, G, K, Q, or S; X₄ can be D, E, G, N, M, Q, S, or W; X₅ can be H or P; X₆ can be G, F, K, N, Q, R, S, or T; and X₇ can be D, I, L, or V.

In some embodiments, an MS peptide can have the general sequence WTRGX₉HX₁₀L (SEQ ID NO:95), wherein X₉ and X₁₀ can be independently any amino acid. In some embodiments, X₉ and X₁₀ can be independently any naturally occurring amino acid. In some embodiments, X₉ can be D, E, G, N, M, Q, S, or W. In some embodiments, X₉ can be S, G, or Q. In some embodiments, X₁₀ can be G, F, K, N, Q, R, S, or T. In some embodiments, X₁₀ can be G, N, or Q. In some embodiments, X₉ can be S, G, or Q and X₁₀ can be, independently of X₉, G, N, or Q.

In some embodiments, an MS peptide can be peptide 1730 (WTRGSHNL: SEQ ID NO:1), 1731 (WTRGGHGL: SEQ ID NO:2),1732 (WTRGQHQL: SEQ ID NO:3) 1733 (WSLGQHRIS: SEQ ID NO:31), 1734 (WIRQGPFDK: SEQ ID NO:32), 1735 (WTRGMHQVS: SEQ ID NO:33), 1736 (WTRSEHNLA: SEQ ID NO:34),1737 (WTLQWHTIQ: SEQ ID NO:35), 1738 (WSLDSHRLV, SEQ ID NO:36), 1739 (WTRGQHKLQ: SEQ ID NO:37), 1740 (WNIKQHSLY: SEQ ID NO:38), 1741 (WTRDQHQLH: SEQ ID NO:39), 1742 (WTLKNHTLS: SEQ ID NO:40),1743 (WTRSMHSLL: SEQ ID NO:41), 1744 (WTRSMHSLV: SEQ ID NO:42),1745 (WTRGDHQVW: SEQ ID NO:43),1746 (WTRGDHQVX (X can be any naturally occurring amino acid): SEQ ID NO:44), 1747 (WTRGMHQVW: SEQ ID NO:45).

In some embodiments, an MS peptide can comprise the formula or sequence X₆₀—X₆₁—P-A-P—X₆₂—X₆₃—X₆₄—X₆₅ (SEQ ID NO: 145), wherein X₆₀ is selected from the group consisting of W, D, E, G, S and A, with W being preferred; X₆₁ selected from the group consisting of L, I, V and a deletion at the position, with L being preferred; X₆₂ is selected from the group consisting of I, L, V and A, with I and L being preferred, and I being particularly preferred; X₆₃ is selected from the group consisting of D and G, with D being preferred; X₆₄ is selected from the group consisting of K, V, T and F, with K and F being preferred, and K being particularly preferred; and X₆₅ is selected from the group consisting of L and P.

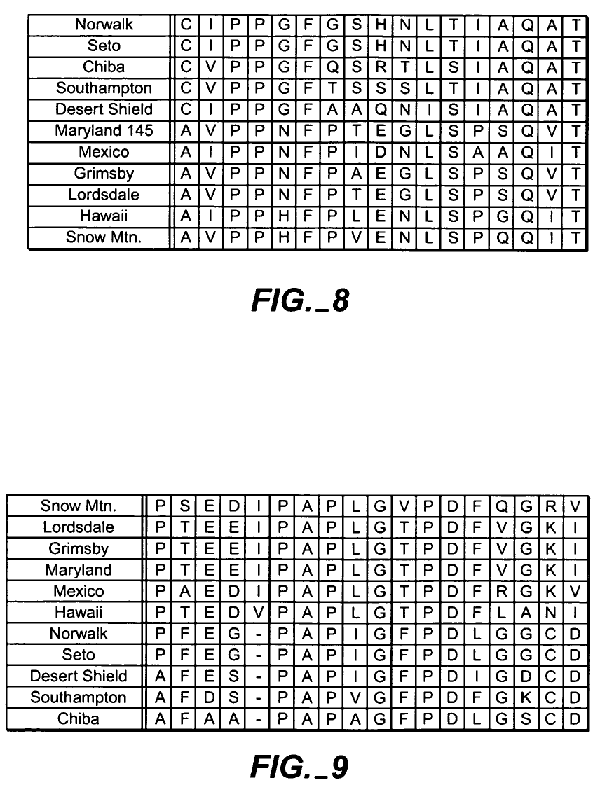

In some embodiments, an MS peptide as exemplified by X₆₀—X₆₁—P-A-P—X₆₂—X₆₃—X₆₄—X₆₅ (SEQ ID NO:145), can, in some embodiments, further comprise (i) one or more amino acids at the amino-terminus, and/or (ii) one or more amino acids at the carboxy-terminus. In some embodiments, an MS peptide as exemplified by X₆₀—X₆₁—P-A-P—X₆₂—X₆₃—X₆₄—X₆₅ (SEQ ID NO:145), can comprise one or more amino acids that corresponds to a Norovirus sequences as depicted in FIG. 9.

In some embodiments, an MS peptide can have the general sequence X₁₁—X₁₂—P-A-P-X₁₃—X₁₄—X₁₅—X₁₆ (SEQ ID NO:46), wherein X₁₁ can be any amino acid; X₁₂ can be an amino acid having a linear or branched alkyl side chain; X₁₃ can be an amino acid having a linear or branched alkyl side chain; X₁₄ can be an amino acid having an acidic or hydrogen side chain; X₁₅ can be an amino acid having a basic, alkyl, or hydroxyalkyl side chain; X₁₆ can be an amino acid having an aliphatic side chain or an imino acid.

In some embodiments, X₁₁ can be any naturally occurring amino acid; X₁₂ can be a naturally occurring amino acid have linear or branched alkyl side chain; X₁₃ can be a naturally occurring amino acid having a linear or branched alkyl side chain; X₁₄ can be a naturally occurring amino acid having an acidic or hydrogen side chain; X₁₅ can be a naturally occurring amino acid having a basic, alkyl, or hydroxyalkyl side chain; and X₁₆ can be a naturally occurring amino acid having an aliphatic side chain or a naturally occurring imino acid.

In some embodiments, X₁₁ can be a naturally occurring amino acid having an acidic side chain; X₁₂ can be an I, L, or V; X₁₃ can be A, I, L, or V; X₁₄ can be D, E, or G; X₁₅ can be an K, T, or V; and X₁₆ can be L or P.

In some embodiments, X₁₁ can be W; X₁₂ can be an I, L, or V; X₁₃ can be I or L; X₁₄ can be D; X₁₅ can be K; and X₁₆ can be L. In some embodiments, X₁₁ can be W; X₁₂ can be L; X₁₃ can be I; X₁₄ can be D; X₁₅ can be an K; and X₁₆ can be L.

In some embodiments, an MS peptide can be peptide 1800 (WLPAPIDKL: SEQ ID NO:4),1801 (DIPAPLGVP: SEQ ID NO:48),1802 (EIPAPLGTP: SEQ ID NO:49),1803 (WIPAPIDKL: SEQ ID NO:50),1804 (WVPAPLDKL: SEQ ID NO:51),1805 (WIPAPLGKL: SEQ ID NO:52),1806 (WVPAPLGKL: SEQ ID NO:53),1807 (WIPAPLGVK: SEQ ID NO:54),1808 (WIPAPLGTL: SEQ ID NO:55),1809 (WVPAPLGTL: SEQ ID NO:56),1810 (WIPAPLGVP: SEQ ID NO:57),1811 (WIPAPLGTP: SEQ ID NO:58), or 1812 (WVPAPLGTP: SEQ ID NO:59); 1812 (WLPAPLDKL: SEQ ID NO:100), 1813 (WIPAPLGVL: SEQ ID NO:101), 1814 (WIPAPLGVL: SEQ ID NO:102),1815 (DIPAPLGTP: SEQ ID NO:103), or 1816 (DVPAPLGTP: SEQ ID NO:104).

In some embodiments, an MS peptide can have the general sequence X₁₈—P-A-P—X₁₉-G-F—P (SEQ ID NO:60), wherein X₁₈ can be any amino acid having an aliphatic or hydroxyalkyl side chain; and X₁₉ can be an amino acid having a linear or branched alkyl side chain.

In some embodiments, X₁₈ can be a naturally occurring amino acid having an aliphatic or hydroxyalkyl side chain; and X₁₉ can be a naturally occurring amino acid having a linear or branched alkyl side chain. In some embodiments, X₁₈ can be A, S, or G; and X₁₉ can be I, V, or A.

In some embodiments, an MS peptide can be peptide 1900 (GPAPIGFP: SEQ ID NO:61),1901 (SPAPIGFP: SEQ ID NO:62),1902 (SPAPVGFP: SEQ ID NO:63),1903 (APAPAGFP: SEQ ID NO:64),1904 (WLPAPIGFL: SEQ ID NO:65),1905 (WLPAPIGFP:SEQ ID NO:66),1906 (WPAPIDKL: SEQ ID NO:67),1907 (WPAPIGKL: SEQ ID NO:68),1908 (WPAPIGFL: SEQ ID NO:69),1909 (WPAPIGFP: SEQ ID NO:70),1910 (WLPAPVDKL: SEQ ID NO:71),1911 (WLPAPVGKL: SEQ ID NO:72),1912 (WLPAPVGFL: SEQ ID NO:73),1913 (WLPAPVGFP: SEQ ID NO:74),1914 (WPAPVDKL: SEQ ID NO:75),1915 (WPAPVGKL: SEQ ID NO:76),1916 (WPAPVGFL: SEQ ID NO:77), 1917 (WPAPVGFP: SEQ ID NO:78),1918 (WLPAPADKL: SEQ ID NO:79),1919 (WLPAPAGKL: SEQ ID NO:80),1920 (WLPAPAGFL: SEQ ID NO:81),1921 (WLPAPAGFP: SEQ ID NO: 82),1922 (WPAPADKL: SEQ ID NO:83),1923 (WPAPAGKL: SEQ ID NO:84),1924 (WPAPAGFL: SEQ ID NO:85),1925 (WPAPAGFP: SEQ ID NO:86), or 1926 (WLPAPIGKL: SEQ ID NO:105).

In some embodiments, an MS peptide can comprise an amino acid sequence that corresponds to the amino acid sequence of another MS peptide. By “corresponds” and grammatical equivalents herein are meant to be homologous or analogous. Therefore, in some embodiments, a first MS peptide corresponds to a second MS peptide or Norovirus protein by having the homology or identity described above with the second MS peptide or Norovirus protein. In some embodiments, a first MS peptide may correspond to a second MS peptide or Norovirus protein but does not have the sequence homology described above with the second MS peptide or Norovirus protein. Therefore, in some embodiments, first and second MS peptides or Norovirus protein may correspond to each other by having analogous sequences, wherein analogy can be established by structural and/or functional relationships. For example, the correspondence between sequences between functionally and/or structurally related proteins and/or peptides can be established for example by comparing the primary structure, e.g., comparing the deduced amino acid sequences of two or more Norovirus ORF2s and an MS peptide. For example, ORF2 of Noroviruses have been shown to encode the viral capsid protein which functions in virus attachment to cells and assembly. Therefore, analogous sequences within the deduced amino acid sequence of ORF2s of Noroviruses can be established. For example, in some embodiments, a first MS peptide may have a sequence that is homologous to the deduced amino acid sequence of a first Norovirus ORF2. By aligning the amino acid sequence of the first Norovirus to a second Norovirus ORF2 amino acid sequence, as described below, a sequence corresponding to the first MS peptide can be identified in the second Norovirus ORF2 (FIGS. 8, 9). In various exemplary embodiments, an analogous or corresponding sequence can be at least about 5 amino acids in length, to at least about 10 amino acids in length, to at least about 20 amino acids in length, and in some embodiments can be longer. In a preferred embodiment, the corresponding sequence are preferably sequential. In some embodiments, a corresponding sequence can be determined by comparing the deduced and/or predicted tertiary structures of two or Norovirus capsid proteins. For example, the region corresponding to amino acids 133 to 137 of NV is within the “S” region, close to the hinge region, of the capsid protein. Thus the invention includes peptides corresponding to this particular area and antibodies that bind to this area.

In some embodiments, an MS peptide can comprise a sequence corresponding to amino acids 133 to 137 (GSHNL: SEQ ID NO:87). Therefore, in some embodiments an MS peptide can comprise peptide 1730. In some embodiments, an MS peptide corresponding to peptide 1730 includes but is not limited to peptide 2000 (WTRAAQNI: SEQ ID NO:88), 2001 (WTRTSSSL: SEQ ID NO:89),2002 (WTRQSRTL: SEQ ID NO:90), 2003 (WTRPVENL: SEQ ID NO:91), 2004 (WTRPLENL: SEQ ID NO:92),2005 (WTRPTEGL: SEQ ID NO:93), 2006 (WTRPAEGL: SEQ ID NO:106), 2007 (WLSPTEGL: SEQ ID NO:107), 2008 (WLSGSHNL: SEQ ID NO:108), 2009 (WIRGSHNL: SEQ ID NO:109), 2010 (WNIGSHNL: SEQ ID NO:110), 2011 (WLSAAQNI: SEQ ID NO:111), 2012 (WIRAAQNI: SEQ ID NO:112), 2013 (WNIAAQNI: SEQ ID NO:113), 2014 (WLSTSSSL: SEQ ID NO:114), 2015 (WIRTSSSL: SEQ ID NO: 115), 2016 (WNITSSSL: SEQ ID NO:116), 2017 (WLSQSTRL: SEQ ID NO:117), 2018 (WIRQSTRL: SEQ ID NO:118), 2019 (WNIQSTRL: SEQ ID NO:119), 2020 (WLSPVENL: SEQ ID NO:120),2021 (WIRPVENL: SEQ ID NO:121),2022 (WNIPVENL: SEQ ID NO:122), 2023 (WLSPLENL: SEQ ID NO:123), 2024 (WIRPLENL: SEQ ID NO:124), 2025 (WNIPLENL: SEQ ID NO:125), 2026 (WIRPTEGL: SEQ ID NO:126), 2027 (WNIPTEGL: SEQ ID NO:127), 2028 (WLSPAEGL: SEQ ID NO:128), 2029 (WIRPAEGL: SEQ ID NO:129), 2030 (WNIPAEGL: SEQ ID NO:130), 2031 (WTRPIDNL: SEQ ID NO:131), 2032 (WLSPIDNL: SEQ ID NO:132), 2033 (WIRPIDNL: SEQ ID NO:133), or 2034 (WNIPIDNL:134). 2035 (WLSQSRTL: SEQ ID NO:135), 2036 (WIRQSRTL: SEQ ID NO:136), 2037 (WNIQSRTL: SEQ ID NO:137); 2038 (WTRPVENI: SEQ ID NO:138), 2039 (WLSPVENI: SEQ ID NO:139), 2040 (WIRPVENI: SEQ ID NO:140), 2041 (WNIPVENI: SEQ ID NO:141).

In some embodiments, an MS peptide can comprise a sequence corresponding to amino acids 319 to 327 of SMV (DIPAPLGVP: SEQ ID NO:48). In some embodiments, an MS peptide can comprise a sequence corresponding to amino acids 320-324 (IPAPL: SEQ ID NO:146) of SMV.

In some embodiments, MS peptides of the present invention can be amino acid sequence variants. These variants fall into one or more of three classes: substitutional, insertional or deletional variants. These variants ordinarily can be prepared by site specific mutagenesis of nucleotides in the DNA encoding an MS peptide, using cassette or PCR mutagenesis or other techniques well known in the art, to produce DNA encoding the variant, and thereafter expressing the DNA in recombinant cell culture as outlined above. However, variant MS peptides having up to about 100-150 residues may be prepared by in vitro synthesis using established techniques. Amino acid sequence variants can be characterized by the predetermined nature of the variation, a feature that sets them apart from naturally occurring variation of the capsid protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed MS peptide variants screened for the optimal combination of desired activity. Techniques for making substitution mutations at predetermined sites in DNA having a known sequence are well known, for example, M13 primer mutagenesis and PCR mutagenesis. Screening of the mutants can be done using assays of capsid protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the MS peptide are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity can be made by selecting substitutions that are less conservative than those shown in Chart I. For example, substitutions may be made which more significantly affect the structure of the polypeptide backbone in the area of the alteration, for example the α-helical or β-sheet structure; the charge or hydrophobicity of the molecule; or the bulk of the side chain. The substitutions which in general are expected to produce the greatest changes in the peptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The variants typically exhibit the same qualitative biological activity, elicit the same immune response, and/or are recognized by the immune response elicited by the naturally-occurring or parental analogue, although variants also are selected to modify the characteristics of the MS peptide as needed. Alternatively, the variant may be designed such that the biological activity of the MS peptide is altered. In general, MS peptides can include 1, 2, or 3 substitutions and/or deletions and/or insertions as compared to the sequences outlined herein, with more substitutions and/or deletions and/or insertions being acceptable or tolerated as the length of the peptide increases.

Covalent modifications of MS peptides are included within the scope of this invention, particularly for screening assays or therapeutic uses. One type of covalent modification includes reacting targeted amino acid residues of MS peptide with an organic derivatizing agent capable of reacting with selected side chains or the N— or C-terminal residues of an MS peptide. Derivatization with bifunctional agents is useful, for instance, for crosslinking MS peptide to a water-insoluble support matrix or surface for use in the methods described below, or for in vivo stability. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

In addition, modifications such as derivitization with polyethylene glycols (and other glycols) to increase the in vivo stability half-life are also included.

MS peptides of the present invention may also be modified in a way to form chimeric molecules comprising an MS peptide fused to another, heterologous polypeptide or amino acid sequence. In a preferred embodiment the MS peptide may be linked to adjutants or other molecules to increase the immune response to the peptide, e.g., immunogens. In an additional embodiment, such a chimeric molecule comprises a fusion of an MS peptide with a tag polypeptide which provides an epitope to which an anti-tag antibody can selectively bind. The epitope tag generally can be placed at the amino-or carboxyl-terminus of the MS peptide (or it may be added to the “new” C-terminus after the hydrophobic amino acid region, generally about 21 residues, is removed). The presence of such epitope-tagged forms of an MS peptide can be detected using an antibody against the tag polypeptide. Also, provision of the epitope tag enables the MS peptide to be readily purified by affinity purification using an anti-tag antibody or another type of affinity matrix that binds to the epitope tag. This also is useful for binding the MS peptide to a support for heterogeneous screening methods. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al, Mol. Cell. Biol. 8:2159-2165 (1988)0; the c-myc tag and the 8F9, 3C7, 6E10, G4 B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA, 87:6393-6397 (1990)). In some embodiments, other fusion partners, generally, but not always proteinaceous are well known; thus all types of fusions, for example, branched and/or linear fusions comprising the peptides of the invention are included.

By “nucleic acid,” “oligonucleotide,” and grammatical equivalents herein are meant at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al., Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see, e.g., Egholm, J. Am. Chem. Soc. 114:1895 (1992); Meier et al., Chem. Int. Ed. Engl. 31:1008 (1992); Nielsen, Nature 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 4,469,863, 5,216,141, 5,386,023, 5,602,240, 5,637,684; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, Carbohydrate Modifications in Antisense Research, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see, e.g., Jenkins et al., Chem. Soc. Rev. (1995) pp. 169-176). Several nucleic acid analogs are described in Rawls, C & E News Jun. 2, 1997, page 35. All of these references are hereby expressly incorporated by reference.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a peptide nucleic acid, each containing a base, are referred to herein as a nucleoside.

By “Norovirus nucleic acid,” “NOR nucleic acid,” and grammatical equivalents herein are meant a nucleic acid comprising a sequence homologous or identical to the positive-sense genomic or full-length genomic RNA packaged into infectious virions, the negative-sense reverse complement of the a Norovirus genomic RNA which serves as a replication intermediate, or a subgenomic length Norovirus RNA of positive or negative polarity, which may or may not be packaged into virions or function as a mRNA. In some embodiments, an Norovirus nucleic acid is about 8-100 nucleotides in length, in a preferred embodiment an Norovirus nucleic acid is about 840 nucleotides in length and an even more preferred embodiment a Norovirus nucleic acid is about 8 to 20 nucleotides in length. As used herein, a nucleic acid is a “Norovirus nucleic acid” if the overall homology of the nucleotide sequence to the nucleotide sequences of a NV is preferably greater than about 75%, more preferably greater than about 80%, even more preferably greater than about 85% and most preferably greater than 90%. In some embodiments, the homology will be as high as about 93 to 95 or 98%. Homology in this context means sequence similarity or identity, with identity being preferred. This homology will be determined using standard techniques known in the art as described below. As used herein, a nucleic acid is a “Norovirus nucleic acid” if it encodes a Norovirus protein as described above. In a preferred embodiment, a Norovirus nucleic acid encodes a Norovirus capsid protein (including capsid peptides) or an MS peptide.

In some embodiments, a Norovirus nucleic acid encodes an MS peptide. In some embodiments, a Norovirus nucleic acid expresses an MS peptide. Thus, in some embodiments a nucleic acid encoding an MS peptide can be functionally linked to a promoter, wherein expression can be constitute and/or inducible, as known in the art. The MS peptide can be expressed either alone or in combination with one or more other proteins, wherein the MS peptide can be expressed as a fusion protein (e.g., phage display, maltose binding protein fusion, etc.). Nucleic acid sequences can be determined by sequencing a nucleic acid expressing an MS peptide as described herein. In some embodiments, a nucleic acid expressing an MS peptide can be synthesized in whole or in part using for example, automated, solid phase synthesis methods, as known in the art. Designing a nucleic acid sequence encoding of an MS peptide is within the abilities of the skilled artisan by reverse translating an MS peptide sequence as disclosed herein to a nucleic acid sequence using the Genetic Code. (Stryer. Biochemistry 15-42 (3d ed. W.H. Freeman & Co. 1988) In some embodiments, the Genetic Code can be the standard Genetic Code. In some embodiments, the Genetic Code can be biased to the codons utilized by yeast, bacteria, mitochondria, etc., or combinations thereof. By way of exemplification and not limitation, a skilled artisan will appreciate that WTRGSHNL (SEQ ID NO: 1) can be encoded by a nucleic acid comprising the nucleotide sequence: 5′-TGG-ACT-CGT-GGT-TCT-CAT-AAT-CTT (SEQ ID NO:94). The skilled also will appreciate that expression required a 5′ codon for methionine (AUG) within the proper sequence context to initiated translation and MS peptide synthesis (e.g., Kozak's rule) and for expression from RNA “T” can be replaced by “U”. (see, e.g., Sambrook et al., Molecular Cloning: A Laboratory Manual (3d. ed. Cold Spring Harbor Laboratory Press)

As is known in the art, a number of different programs can be used to identify whether a protein (or nucleic acid as discussed herein) has sequence identity or similarity to a known sequence. Sequence identity and/or similarity is determined using standard techniques known in the art, including, but not limited to, the local sequence identity algorithm of Smith and Waterman, Adv. Appl. Math. 2:482 (1981), by the sequence identity alignment algorithm of Needleman & Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, Current Methods in Sequence Comparison and Analysis in Macromolecule Sequencing and Synthesis, Selected Methods and Applications, pp. 127-149, Alan R. Liss, Inc. (1988).

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, J. Mol. Evol. 35:351-360 (1987); the method is similar to that described by Higgins and Sharp, CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215,403-410, (1990) and Karlin et al., Proc. Natl. Acad. Sci. USA 90:5873-5787 (1993). A particularly useful BLAST program is the WU-BLAST-2 program which was obtained from Altschul et al., Methods in Enymology 266: 460-480 (1996) (http://blast.wustl/edu/blast/README.html). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and X₅₆ set to 40 for database search stage and to 67 for the output stage of the algorithms. Gapped alignments are triggered by a score corresponding to ˜22 bits.

A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

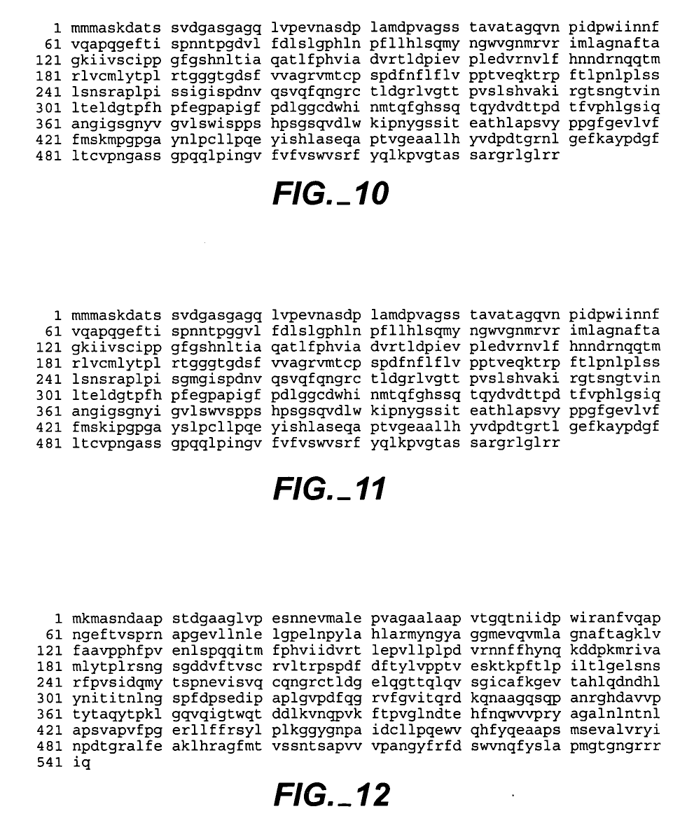

The alignment may include the introduction of gaps in the sequences to be aligned. In addition, for sequences which contain either more or fewer amino acids than the amino acid sequences depicted in FIGS. 8, 9, 10, 11, and 12, it is understood that in one embodiment, the percentage of sequence identity will be determined based on the number of identical amino acids in relation to the total number of amino acids. Thus, for example, sequence identity of sequences shorter than that of the sequence depicted in FIGS. 8, 9, 10, 11, and 12 will be determined using the number of amino acids in the shorter sequence. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

By “antibody” and grammatical equivalents herein are meant polyclonal and monoclonal antibody (MAb). Methods of preparation and purification of monoclonal and polyclonal antibodies are known in the art and, for example, are described in Harlow and Lane, Antibodies: A Laboratory Manual (New York: Cold Spring Harbor Laboratory Press, 1988). By “MS antibody” and grammatical equivalents thereof include an antibody that binds to a Norovirus, a Norovirus protein, or MS peptide. The binding of an MS antibody to an MS peptide preferably blocks or inhibits binding of the MS antibody to a Norovirus. In other embodiments, an MS antibody preferably inhibits binding of a Norovirus to a cell, including but not limited to a host cell or an erythrocyte (RBC) in vitro and/in vivo. In some embodiments, an MS antibody competes with another antibody for binding to a Norovirus or an MS peptide. In some embodiments, an MS antibody neutralizes NV infectivity. Thus, by “neutralization,” “neutralize,” “neutralizing” and grammatical equivalents herein is meant to inhibit or lessen the infective capacity or ability of a Norovirus. In another embodiment an MS antibody protects a host from Norovirus infection or disease, with disease being preferred. Preferred MS antibodies include NV54.6, NV72.10, and/or SMV62.21, described in the examples. Excluded from the definition of MS antibody is NV8812 as disclosed by White et al. J. Virol. 70:6589-6597 (1996).

In some embodiments, MS antibodies can be generated by immunization with an Norovirus capsid protein and/or a Norovirus VLP comprising a capsid protein. In some embodiments, MS antibodies can be generated by immunization with an MS peptide. When an MS peptide is used to generate MS antibodies, the MS peptide can share at least one epitope or antigenic determinant with the full length capsid protein. Accordingly, epitopes or determinants may be linear or conformational, as described above. In most instances, antibodies made to a MS peptide that is smaller than the full length protein can bind to the full length protein. In a preferred embodiment, the epitope can be unique; that is, antibodies can be generated to a unique epitope show little or no cross-reactivity to other proteins of other Noroviruses in the same and/or different genogroup and/or genetic cluster. Thus, in some embodiments, the an MS

In a preferred embodiment, MS antibodies are provided. MS antibodies may be polyclonal or monoclonal with the latter being preferred. In a preferred embodiment, MS antibodies to Norovirus capsid can be capable of reducing or eliminating the biological function of a Norovirus capsid protein, as described below. That is, the addition of MS antibodies (either polyclonal or preferably monoclonal) to a Norovirus (or cells containing a Norovirus) may decrease or eliminate Norovirus infectivity, binding to a host cell, or virus yield. Generally, at least about a 25% decrease is preferred, with at least about 50% being particularly preferred and at least about a 95-100% decrease being especially preferred.

MS monoclonal antibodies are directed against a single antigenic site or a single determinant on an antigen. Thus, MS monoclonal antibodies, in contrast to polyclonal antibodies, which are directed against multiple different epitopes, are very specific. MS monoclonal are usually obtained from the supernatant of hybridoma culture (see Kohler and Milstein, Nature 256:495-7 (1975); Harlow and Lane, Antibodies: A Laboratory Manual (New York: Cold Spring Harbor Laboratory Press, 1988).

In a preferred embodiment, MS antibodies are humanized. Using current monoclonal antibody technology one can produce a humanized antibody to virtually any target antigen that can be identified. (Stein, Trends Biotechnol. 15:88-90 (1997)) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin and all or substantially all of the FR regions can be those of a human immunoglobulin consensus sequence. The humanized antibody optimally also can comprise at least a portion of an immunoglobulin constant region (Fc), typically that of a human immunoglobulin. (Jones et al., peptide epitope generates antibodies that are cross-reactive to other Noroviruses. In some embodiments, an MS peptide induces a neutralizing immune response, e.g., antibody, to a Norovirus. In some embodiments, an MS peptide induces an immune response that inhibits Norovirus binding to cell, including but not limited to a host cell, a CaCo-2 cell, or an erthrythrocyte (RBC).

The terms “antibody” and “MS antibody,” include antibody fragments, as are known in the art, such as Fab, Fab′, F(ab′)2 or other antigen-binding subsequences of antibodies, such as, single chain antibodies (Fv for example), chimeric antibodies, etc., either produced by the modification of whole antibodies or those synthesized de novo using recombinant DNA technologies. The term “antibody” further comprises polyclonal antibodies and MAbs which can be agonist or antagonist antibodies.

MS antibodies of the invention specifically bind to Norovirus capsid proteins or MS peptides. By “specifically bind” herein is meant that the MS antibodies have a binding constant in the range of at least 10⁻⁴-10⁻⁶ M⁻¹, with a preferred range being 10⁻⁷-10⁻⁹ M⁻¹. Thus, in a preferred embodiments, MS antibodies block the binding of a second antibody to Norovirus or MS antibodies block the binding of Norovirus to a cell. By “blocking,” “inhibiting” and grammatical equivalents herein includes binding of MS antibody to Norovirus reduces the amount of Norovirus that binds to a host cell or second antibody, particularly an antibody such as NV54.6, NV72.10, and SMV61.21. In some embodiments, blocking occurs because the MS antibody and the second antibody (e.g., NV54.6) or the MS antibody and cell recognize the same epitope or region on a Norovirus protein. In some embodiments, blocking occurs because the MS antibody and the second antibody or the MS antibody and cell recognize distinct but spatially related epitopes or regions on a Norovirus. Thus, in a preferred embodiment, the inhibition is competitive. In an alternative embodiment, the inhibition is noncompetitive although this is generally not preferred. Generally, at least about 25% inhibition is preferred, with at least about 50% being particularly preferred and at least about a 95-100% inhibition being especially preferred.

In a preferred embodiment, an MS peptide of the present invention may be identified by its immunological activity, e.g., its ability to bind to an MS antibody specific for a linear or conformational epitope. The term “immunological activity” means the ability of an MS peptide to cross react with an MS antibody and/or to induce the production of an MS antibody. Thus, for example, a protein is an MS peptide if it displays the immunological activity of a protein comprising a Norovirus capsid protein. Nature 321:522-525 (1986); Riechmann et al., Nature 332:323-329 (1988); and Presta, Curr. Op. Struct. Biol. 2:593-596 (1992))

Methods for humanizing non-human antibodies are well known in the art. Generally, a humanized antibody has one or more amino acid residues introduced into it from a source which is non-human. These non-human amino acid residues are often referred to as import residues, which typically can be taken from an import variable domain. Humanization can be essentially performed following the method of Winter and co-workers (Jones et al., supra; Riechmann et al., supra; and Verhoeyen et al., Science 239:1534-1536 (1988)), by substituting rodent CDRs or CDR sequences for the corresponding sequences of a human antibody. Additional examples of humanized murine monoclonal antibodies are also known in the art, e.g., antibodies binding human protein C (O'Connor et al., Protein Eng. 11:321-328 (1998)), interleukin 2 receptor (Queen et al., Proc. Natl. Acad. Sci., U.S.A. 86:10029-33 (1989)), and human epidermal growth factor receptor 2 (Carter et al., Proc. Natl. Acad. Sci. U.S.A. 89:4285-9 (1992)). Accordingly, such humanized antibodies are chimeric antibodies (U.S. Pat. No. 4,816,567), wherein substantially less than an intact human variable domain has been substituted by the corresponding sequence from a non-human species. In practice, humanized antibodies typically can be human antibodies in which some CDR residues and possibly some FR residues can be substituted by residues from analogous sites in rodent antibodies.

Human antibodies also can be produced using various techniques known in the art, including phage display libraries. (Hoogenboom and Winter, J. Mol. Biol. 227:381 (1991); Marks et al., J. Mol. Biol. 222:581 (1991)) The techniques of Cole et al. and Boerner et al. are also available for the preparation of human monoclonal antibodies. (Cole et al., Monoclonal Antibodies and Cancer Therapy, Alan R. Liss, p. 77 (1985) and Boemer et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production can be observed, which closely resembles that seen in humans in all respects, including gene rearrangement, assembly, and/or antibody repertoire. This approach is described, for example, in U.S. Pat. Nos. 5,545,807, 5,545,806, 5,569,825, 5,625,126, 5,633,425, 5,661,016, and in the following scientific publications: Marks et al., Bio/Technology 10:779-783 (1992); Lonberg et al. Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg and Huszar, Intern. Rev. Immunol. 13:65-93 (1995).

Once made, the MS compositions of the invention (e.g., antibodies and peptides) find use in a number of applications. In general, MS antibodies and MS peptides can find use in inhibiting the interaction of Norovirus with cells. In some embodiments, MS antibodies and MS peptides can find use in inhibition the interaction of NV and/or SMV with cells. Thus, particularly preferred are therapeutic treatments, as outlined below. In addition, these compositions find use in diagnostic assays and kits to detect the presence of Norovirus in a subject, patient, or sample. Furthermore, the compositions of the invention can be used to discover additional antibodies and peptides which compete for binding with the MS compositions. Thus, screening assays, generally but not always competitive screening assays, particularly high throughput screening assays, can also be done. For example, an MS component of the invention may be attached to a solid support and binding components can be evaluated.

In a preferred embodiment, the MS compositions of the invention find use in the treatment of Norovirus disease. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

In a preferred embodiment, MS antibodies of the present invention that bind to the capsid protein and prevent Norovirus attachment to host cells are administered to patient in a therapeutically effective amount. By “therapeutically effective amounts” herein is meant an amount of antibody which is sufficient to ameliorate Norovirus disease. This amount may be different depending on whether prophylactic or therapeutic treatment is desired. Determining the dosages and times of administration for a therapeutically effective amount are well within the skill of the ordinary person in the art. These amounts may be adjusted depending on the severity of disease or susceptibility of the patient.

In a preferred embodiment, MS peptides of the present invention find use as immunogens, vaccines, and antiviral compounds. Therefore, in some embodiments a peptide can be formulated to be suitable as an immunogen and/or a therapeutic administration to a patient, host, and/or subject.

By “vaccine” herein is meant an antigen or compound which elicits an immune response in a patient. The vaccine may be administered prophylactically, for example to a patient never previously exposed to the antigen, such that subsequent infection by a Norovirus is prevented. Alternatively, the vaccine may be administered therapeutically to a patient previously exposed or infected by a Norovirus. While, in some embodiments, infection cannot be prevented, an immune response can be generated which allows the patient's immune system to more effectively combat the infection. Thus, for example, there may be a decrease or lessening of the symptoms associated with infection. In a preferred embodiment, a Norovirus comprises MS peptides that induce an immune response to a various types of Noroviruses.

By “immune response” and grammatical equivalents herein are meant a response by a host's or patient's cells of the immune system to an antigen which the immune cells recognize as being a foreign antigen or an antigen not normally detected in the host. In some embodiments, the immune response is an antibody response. By “antibody response” and grammatical equivalents herein are meant the response of the immune system of a host or patient to an antigen, e.g., vaccine or MS peptide, that results in the production by the host's or patient's immune system of antibody that binds to the antigen. Determining the dose and immunization schedule to induce an immune response in a subject is within the abilities of the skilled artisan.

The administration of an MS peptide as a vaccine can be accomplished in a variety of ways, e.g., parenteraly or mucosally, e.g., oral, nasal, rectal administration. Generally, the MS peptides can be formulated according to known methods to prepare pharmaceutically useful compositions, whereby therapeutically effective amounts of MS peptide can be combined in admixture with a pharmaceutically acceptable carrier vehicle. Suitable vehicles and their formulation are well known in the art. Such compositions can contain pharmaceutically effective amount of MS peptide together with a suitable amount of vehicle in order to prepare pharmaceutically acceptable compositions for effective administration to a patient. The composition may include salts, buffers, carrier proteins such as serum albumin, targeting molecules to localize MS peptides at the appropriate site or tissue within the patient, and other molecules. The composition may include adjuvants as well. The formulation is chosen at the discretion of the practitioner and is dependent on the route of immunization, age and immune status of the patient, and severity of disease.

Where sustained-release administration of an MS peptide is desired in a formulation with release characteristics suitable for the treatment of any disease or disorder requiring administration of the MS peptide, microencapsulation of the polypeptide is contemplated. Microencapsulation of recombinant proteins for sustained release has been successfully performed with human growth hormone (rhGH), interferon-(rhIFN), interleukin-2, and MN rgp120. (Johnson et al., Nat. Med. 2:795-799 (1996); Yasuda, Biomed. Ther. 27:1221-1223 (1993); Hora et al., Bio/Technology 8:755-758 (1990); Cleland, Design and Production of Single Immunization Vaccines Using Polylactide Polyglycolide Microsphere Systems in Vaccine Design: The Subunit and Adjuvant Approach, 439-462 (Powell and Newman, eds. Plenum Press 1995); WO97/03692, WO96/40072, WO96/07399; and U.S. Pat. No. 5,654,010. The sustained-release formulations of polypeptides were developed using poly-lactic-coglycolic acid (PLGA) polymer due to its biocompatibility and wide range of biodegradable properties. The degradation products of PLGA, lactic, and glycolic acids, can be cleared quickly within the human body. Moreover, the degradability of this polymer can be adjusted from months to years depending on its molecular weight and composition. Lewis, Controlled release of bioactive agents from lactide/glycolide polymer in Biodegradable Polymers as Drug Delivery Systems 1-41 (Chasin and Langer eds. Marcel Dekker 1990).

“Pharmaceutically acceptable salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucuronic or galacturonic acids and the like (see, e.g., Berge et al., 1977, J. Pharm. Sci. 66: 1-19).

“Pharmaceutically acceptable vehicle” refers to a diluent, adjuvant, excipient or carrier with which a compound of the invention is administered.

“Pharmaceutically effective amount” or “therapeutically effective amount” refers to an amount sufficient to produce the desired physiological effect or amount capable of achieving the desired result, particularly for treating the disorder or disease condition, including reducing or eliminating one or more symptoms of the disorder or disease or prevention of the disease or condition. Accordingly, in a preferred embodiment, vaccines induce an immune response that reduces or eliminates one or more symptoms of Norovirus disease or prevents Norovirus disease or condition. Generally, this ranges from about 0.001 mg to about 1 gm, with a preferred range of about 0.05 mg. These amounts may be adjusted if adjuvants are used.

In a preferred embodiment, the compositions of the invention are antiviral compounds. By “antiviral” and grammatical equivalents herein are meant a compound that inhibits the replication cycle of a NV. The MS peptide may be administered prophylactically, for example to a patient never previously exposed to NV, such that subsequent infection by NV is prevented. Alternatively, MS peptide may be administered therapeutically to a patient previously exposed or infected by NV. MS peptides compounds may be administered per se but can be typically formulated and administered in the form of a pharmaceutical composition. The exact composition can depend upon, among other things, the method of administration, such as orally or parenterally, and can be apparent to those of skill in the art. A wide variety of suitable pharmaceutical compositions are described, for example, in Remington's Pharmaceutical Sciences, 20th ed. (2001).

Formulations suitable for oral administration can consist of (a) liquid solutions, such as an effective amount of the active compound suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are the preferred methods of administration. A specific example of a suitable solution formulation may comprise from about 0.5-100 mg/ml compound and about 1000 mg/ml propylene glycol in water. Another specific example of a suitable solution formulation may comprise from about 0.5-100 mg/ml compound and from about 800-1000 mg/ml polyethylene glycol 400 (PEG 400) in water.

A specific example of a suitable suspension formulation may include from about 0.5-30 mg/ml compound and one or more excipients selected from the group consisting of: about 200 mg/ml ethanol, about 1000 mg/ml vegetable oil (e.g., corn oil), about 600-1000 mg/ml fruit juice (e.g., grapefruit juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml compound, about 100-200 mg/ml lecithin (or other phospholipid or mixture of phospholipids) and optionally about 5 mg/ml cholesterol in water. For subcutaneous administration of certain PBI compounds, a liposome suspension formulation including 5 mg/ml compound in water with 100 mg/ml lecithin and 5 mg/ml compound in water with 100 mg/ml lecithin and 5 mg/ml cholesterol provides good results.

The formulations of compounds can be presented in unit-dose or multi-dose sealed containers, such as ampules and vials. Injection solutions and suspensions can be prepared from sterile powders, granules, and tablets of the kind previously described.

The pharmaceutical preparation can be preferably in unit dosage form. In such form, the preparation can be subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail below.

In therapeutic use for the treatment of Norovirus infection, the MS compositions (e.g., antibodies and peptides) utilized in the pharmaceutical method of the invention can be administered to patients diagnosed with Norovirus infection at dosage levels suitable to achieve therapeutic benefit. By “therapeutic benefit” and grammatical equivalents are meant the administration of the compound leads to a beneficial effect in the patient over time. For example, therapeutic benefit can be achieved when the Norovirus titer or load in a patient is either reduced or stops increasing. Therapeutic benefit also can be achieved if the administration of a compound slows or halts altogether the onset of adverse symptoms that typically accompany Norovirus infections, regardless of the Norovirus titer or load in the patient.

The MS peptides and/or compositions thereof may also be administered prophylactically in patients who are at risk of developing Norovirus infection, or who have been exposed to Norovirus, to prevent the development of Norovirus infection. For example, the MS peptides and/or compositions thereof may be administered patient likely to have been exposed to Norovirus.

The present invention further provides methods of blocking MS antibody binding to a Norovirus. In one embodiment, an unlabelled MS antibody binds a Norovirus and blocks the binding of a labeled antibody. In an alternative embodiment, a labeled MS antibody can be inhibited from binding to a Norovirus by an unlabeled antibody. The percent inhibition is calculated by the decrease of labeled-antibody binding in the presence of unlabeled antibody. The present invention further provides methods of blocking MS antibody binding to a Norovirus by use of an MS peptide. In a preferred embodiment, a labeled MS antibody binds an MS peptide which blocks the binding of the MS antibody to a Norovirus. The percent inhibition can be calculated by the decrease of MS antibody binding in the presence as compared to the absence of the MS peptide. The present invention further provides a method of blocking Norovirus binding to a cell, including but not limited to a cell that can be productively infected with a Norovirus (i.e., a host cell) to produce infectious virus. In a preferred embodiment, host cells, preferably differentiated CaCo-2 cells, can be treated with an MS peptide which inhibits binding of labeled Norovirus, e.g., recombinant Norovirus VLPs to the cells.

A compound, such as an MS antibody, MS peptide or Norovirus, can be directly or indirectly conjugated to a label which provides a detectable signal, e.g., radioisotope, fluorescers, enzyme, antibodies, particles, such as but not limited to, magnetic particles, chemiluminescers, or specific binding molecules, etc. Specific binding molecules include binding pairs, such as biotin and streptavidin, digoxin and antidigoxin etc. Preferred labels include, but are not limited to, fluorescent labels, label enzymes, and radioisotopes.

In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles. In a preferred embodiment, the detection label can be a primary label. A primary label can be directly detected, including but not limited to, a fluorophore.

Preferred labels include chromophores or phosphors but can be preferably fluorescent dyes or moieties. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705 and Oregon green. Suitable optical dyes are described in Molecular Probes Handbook by Richard P. Haugland (1996), hereby expressly incorporated by reference. Suitable fluorescent labels also include, but are not limited to, green fluorescent protein (GFP; Chalfie, et al., Science 263(5148):802-805 (1994); and EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, Biotechniques 24(3):462-471 (1998); Heim, et al., Curr. Biol. 6:178-182 (1996)), enhanced yellow fluorescent protein (EYFP, Clontech Laboratories, Inc.), luciferase (Ichiki, et al., J. Immunol. 150(12):5408-5417 (1993)), β-galactosidase (Nolan, et al., Proc Natl Acad Sci USA 85(8):2603-2607 (1988)) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

Particularly preferred labels for use in the present invention include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC and quantitation of fluorescent probe conjugation may be assessed to determine degree of labeling are known in the art.

In another preferred embodiment, the fluorescent label can be a GFP and, more preferably, a Renilla, Ptilosarcus, or Aequorea species of GFP.

In a preferred embodiment, a secondary detectable label can be used. A secondary label is one that can be indirectly detected. For example, a secondary label can bind or react with a primary label for detection, can act on an additional product to generate a primary label (e.g.,enzymes) etc. Secondary labels include, but are not limited to, one of a binding partner pair; chemically modifiable moieties; nuclease inhibitors, enzymes such as horseradish peroxidase, alkaline phosphatases, lucifierases, etc.

In a preferred embodiment, the secondary label can be a binding partner pair. For example, the label may be a hapten or antigen, which can bind its binding partner. For example, suitable binding partner pairs include, but are not limited to, antigens (such as proteins (including peptides) and small molecules) and antibodies (including fragments thereof (FAbs, etc.)); proteins and small molecules (including biotin/streptavidin); enzymes and substrates or inhibitors; other protein-protein interacting pairs; receptor-ligands; and carbohydrates and their binding partners, e.g., lectins. Nucleic acid—nucleic acid binding proteins pairs also can be useful. Preferred binding partner pairs include, but are not limited to, biotin (or imino-biotin) and streptavidin, digeoxinin and Abs, and Prolinx reagents (Cambrex Biosciences).

In a preferred embodiment, the binding partner pair comprises an antigen and an antibody that will specifically bind to the antigen. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

In a preferred embodiment, the secondary label can be a chemically modifiable moiety. In this embodiment, labels comprising reactive functional groups are incorporated into the molecule to be labeled. The functional group can then be subsequently labeled (e.g., either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see Pierce Chemical Company catalog, Technical section on cross-linkers, pp. 155-200 (1994), incorporated herein by reference). The type of label is chosen at the discretion of the practitioner and includes, for example, enzymatic, radioactive, and fluorescent labels. (see Haugland. Handbook of Fluorescent Probes and Research Chemicals. 6^{th} ed. Molecular Probes, Eugene, Oreg.).

The present invention further provides kits for use within any of the above compositions and methods. Such kits typically comprise two or more components necessary for performing a diagnostic assay. Components may be compounds, reagents, containers and/or equipment. For example, one container within a kit may contain an MS antibody that specifically binds to a Norovirus and finds use in the identification of a Norovirus isolate from a clinical sample. Such antibodies may be provided attached to a label, as described above. One or more additional containers may enclose elements, such as reagents or buffers, to be used in the assay. Such kits may also, or alternatively, contain a detection reagent as described above that contains a reporter group suitable for direct or indirect detection of antibody binding. Alternatively, a kit may be designed to detect Norovirus antibody in a biological sample, such feces or serum. Such kits generally comprise at least one MS peptide, as described above, that binds to anti-Norovirus antibody. Such an MS peptide finds use, for example, in the detection of anti-Norovirus antibody in a clinical sample.

In the present application, use of the singular includes the plural unless specifically stated otherwise. All literature and similar materials cited in this application, including but not limited to patents, patent applications, articles, books, and treatises regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls. Aspects of the present disclosure may be further understood in light of the following examples, which should not be construed as limiting the scope of the present disclosure in any way.

### 6. EXAMPLES

### Example 1

### MAbs NV54.6 and NV72.10

MAbs NV54.6 and 72.10 were generated by immunizing Balb/c mice with purified Norwalk virus VLPs (rNV). Each mouse received 2 intraperitoneal immunizations of 500 μg rNV. Hybridomas secreting antibody to rNV particles were screened by dot blot. Hybridoma supernatants positive for reactivity were further selected for their ability to block radioactively labeled rNV binding to human intestinal CaCo-2 cells in culture. NV54.6 and NV72. 10 were both determined to be IgG1's the Boerhinger Mannhiem Isotyping Kit.

CaCo-2 cells were grown in Earle's minimum essential medium (MEM), supplemented with 10% fetal bovine serum (FBS), L-glutamine, MEM nonessential amino acids, N-2-hydroxyethylpiperazine-N′-2-ethanesulfonic acid (HEPES) buffer, penicillin, and streptomycin in a 5% CO₂ incubator. At 7 to 14 days postconfluency, CaCo-2 cells showed biochemical and morphologic markers of differentiation (2.3 to 2.8 μmol/mg/h sucrase activity in the presence of domes) and were considered differentiated cultures (D-CaCo-2).

Recombinant Norwalk virus (rNV) VLPs were prepared by infecting Sf9 insect cells (3×10⁶ cells/ml) at a multiplicity of infection (m.o.i.) of 10 PFU/cell with baculovirus recombinant Bac-rNV C8, which expresses Norwalk virus (NV) capsid protein (Jiang et al. 1992. Expression, self-assembly, and antigenicity of the Norwalk virus capsid protein. J. Virol. 66:6527:6532). Metabolically radiolabeled rNVs were prepared by placing the cells in methionine-free Grace's medium at 28 h postinfection (hpi) for 30 min. and adding 25 to 30 μCi of [³⁵S]methionine (Trans-³⁵ S-label; ICN, Irvine, Calif.)/ml. At 4 to 6 h postlabeling, 50 μg unlabeled methionine/ml was added. Cultures were harvested at day 7 postinfection, cells were pelleted, and rNVs released into the medium were purified as described by White et al. 1996. Attachment and entry of recombinant Norwalk virus capsids to cultured human and animal cells. J. Virol. 70:6589-6597.

For rNP binding assays, CaCo-2 cells were cultured 7 to 14 days postconfluency in 24- or 96-well plates (Costar, Cambridge, Mass.). Cell monolayers were washed three times with cold PBS or serum-free Eagle's minimum essential medium containing 1% bovine serum albumin, fraction V (BSA; Calbiochem, La Jolla, Calif.) and chilled to 4° C. Purified radiolabeled rNVs were added to duplicate wells in scrum-free medium-1% BSA at fmal volumes of 200 μl/well in 24-well plates or 30 μl/well in 96-well plates. Plates were incubated for 1 h with gentle agitation at 4 C to inhibit internalization. Binding reaction was terminated by washing the cells three times with cold PBS containing 0.1% BSA and lysed with radioimmunoprecipitation assay (RIPA) buffer (0.15 M NaCl, 1% sodium deoxycholate, 1% Triton X-100, 0.1% SDS, 1% trasylol, 10 mM Tris-HCl [pH 7.2]). Total radioactivity in the sample was determined by liquid scintillation spectrometry. The number of cells/well was determined by counting trypsinized cells from triplicate wells.

For rNV hemagglutination assays, Group O, Type Negative (O⁻) whole blood was collected and suspended in 2 volumes Alsever's solution (2.05% glucose [w/v], 0.8% sodium citrate, 0.055% citric acid, and 0.42% sodium chloride; pH 6.1) and stored at 4° C. until use. The red blood cells (RBCs) were washed and packed by diluting 1 ml cells in 14 ml PBS-cmf (Calcium-Magnesium Free, pH 7.4) and centrifuged for 15 min at 500×g. Directly before the assay was performed, packed RBCs were resuspended in 0.85% saline at 0.5% and stored on ice (e.g., 25 μl packed RBCs to 5 ml 0.85% saline).

Purified rNV VLPs were serially diluted (2-fold) in PBS-H (0.01 M sodium phosphate, 0.15 M sodium chloride, pH5.5; sterile filtered using 0.2 μm pore filter) on ice, starting at about 500 mg/ml. Dilutions were then added to corresponding wells of a 96 well V-bottom plate at 50 μl per well. An equal volume (50 μl) 0.5% RBCs in 0.85% saline were added to the wells containing serially diluted VLPs. The plates were gently mixed, covered, and incubated at 4° C. for approximately 2 hrs, or until a row containing RBCs and PBS-H only had settled.

To determine if MAbs NV54.6 and NV72. 10 blocked VLP hemagglutination, partially purified MAbs were diluted to 2 μg/μl in PBS-H and 1 μl MAb dilution was added to each serial dilution of VLPs. The HA assay was performed as above and settling of RBCs that contained VLPs and MAbs were compared with positive HA controls. The results indicated that NV54.6 inhibited rNV hemagglutination up to 62.5 μg/ml. MAb 72.10 inhibited rNV hemagglutination up to 250 μg/ml.

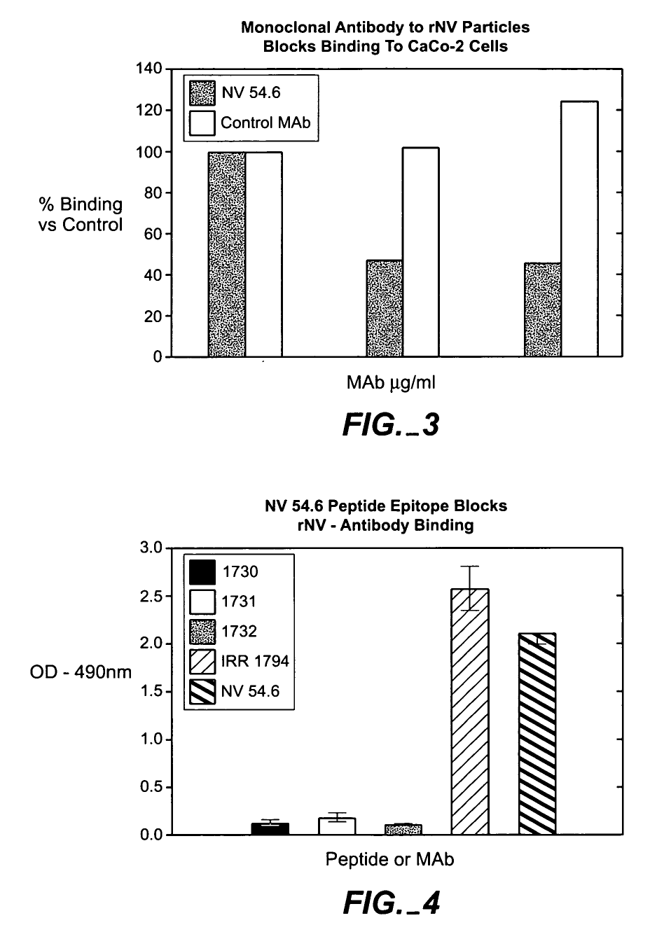

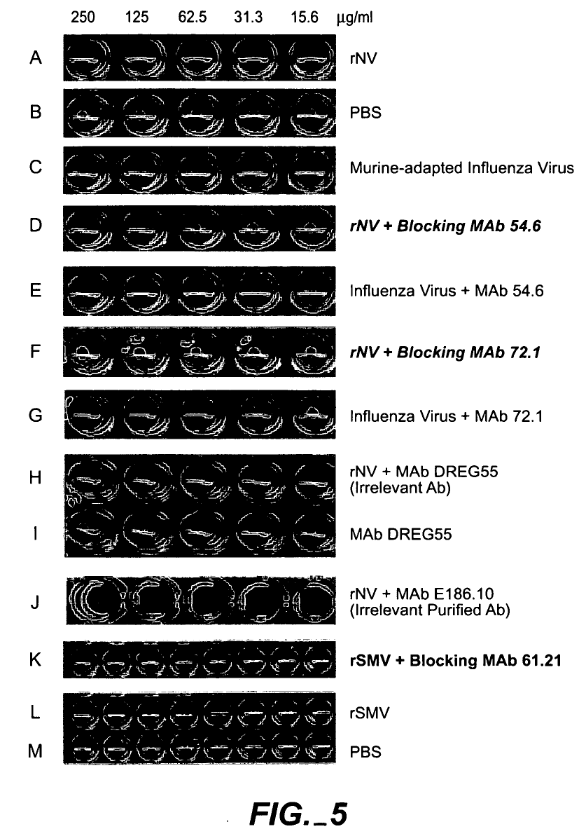

To demonstrate NV54.6 blocking of rNVs to CaCo-2 cells, radiolabeled rNVs (15 μg/10⁵ cells) were mixed with serial dilutions of purified NV54.6 in 0.01 M PBS (final volume, 20 μl) for 1 h at 37° C. This mixture was chilled on ice, 10 μl 3% BSA in serum-free MEM was added to each reaction mixture, and added to confluent monolayers in 96-well plates that had been prewashed with cold serum-free MEM-1% BSA. A Norovirus non-reactive antibody, DREG was used as an isotype matched negative control. Binding was assayed as described above. The results shown in FIG. 3 demonstrate that purified NV54.6 blocks the binding of rNVs to CaCo-2 cells in a dose-dependent manner.

By screening a phage display library, NV54.6 and NV72.10 were found to peptides 1734 (WIRQGPFDK: SEQ ID NO:5), 1735 (WTRGMHQVS: SEQ ID NO:6), 1736 (WTRSEHNLA: SEQ ID NO:8), 1737 (WTLQWHTIQ: SEQ ID NO:9), 1738 (WSLDSHRLV, SEQ ID NO:10), 1739 (WTRGQHKLQ: SEQ ID NO:11), 1740 (WNIKQHSLY: SEQ ID NO:13), 1741 (WTRDQHQLH: SEQ ID NO:14),1742 (WTLKNHTLS: SEQ ID NO: 16), 1743 (WTRSMHSLL: SEQ ID NO:17), 1744 (WTRSMHSLV: SEQ ID NO:18), 1745 (WTRGDHQVW: SEQ ID NO:19),1746 (WTRGDHQVX (X can be any amino acid)): SEQ ID NO:20), and 1747 (WTRGMHQVW: SEQ ID NO:21). Comparing the amino acid sequences of the identified peptides yielded consensus sequences, e.g., W—X₁—X₂—X₃—X₄—X₅—X₆—X₇—X₈ and WTRGXHXL (SED ID NO:96). Peptides 1730, 1731, and 1732 were synthesized to conform with these consensus sequences and were found to recognized by NV54.6 and NV72.10.

The epitope recognized by NV54.6 and NV72.10 was found to be a conformational epitope by analyzing their reactivity with completely denatured (boiled, SDS and β-ME treated) rNV capsid protein by SDS-PAGE and Western immunoblot. NV 54.6 and NV72.10 reacted only with rNV capsid protein that had not been denatured prior to electrophoresis. NV54.6 and NV72.10 also did not react with VLPs of other Noroviruses (TV, SMV, DSV, MV, HV, SHV, LV) tested by a non-denaturing dot blot (see, FIG. 7).

A comparison of the amino acid sequences of the 1730, 1731, and 1732 demonstrated that peptide 1730 has five amino acids identical to amino acids 133-137 of NV capsid protein, which comprises the motif, GXHXL (SEQ ID NO:47). (FIG. 8). This sequence was found within a conserved region of NV and other Norovirus VP 1 s but itself was not well conserved among the GI and GII Noroviruses (FIG. 8), which is consistent with the reactivity of NV54.6 and NV72.10 with these other viruses. This five amino acid sequence resides in a loop of the S domain of NV capsid protein between βE and an α-helix, adjacent to the hinge region. (Prasad et al., Science 286:287-290).

### Example 2

### MAb 61.21

MAb SMV61.21 was generated according to the procedure in Example 1, with the exception that recombinant Snow Mountain virus (rSMV) VLPs were used as the immunogen. rSMV VLPs were made as described in Lochridge et al. Virus Genes 26:71-82 (2003). A peptide recognized by SMV62.21 identified by phage display as described above was found to have the sequence: WLPAPIDKL (1800, SEQ ID NO:4). This epitope is partially conserved among GI and GII Noroviruses VP1s (FIG. 9). The corresponding PAP sequence of NV is part of a small loop between β strands βEf2 and βEf3 of the capsid protein. (Prasad et al. Science 286:287-290 (1999)). White et al. indicated this region may be important in attachment and entry of NV to cultured human and animal cells. (J. Virol. 70:6589-6597). This interpretation is consistent with the ability of SMV61.21 to inhibite rSMV VLP hemagglutination. The hemagglutination inhibition assay was performed as described above with the exception that Group A, Type Positive (A+) RBCs were used.

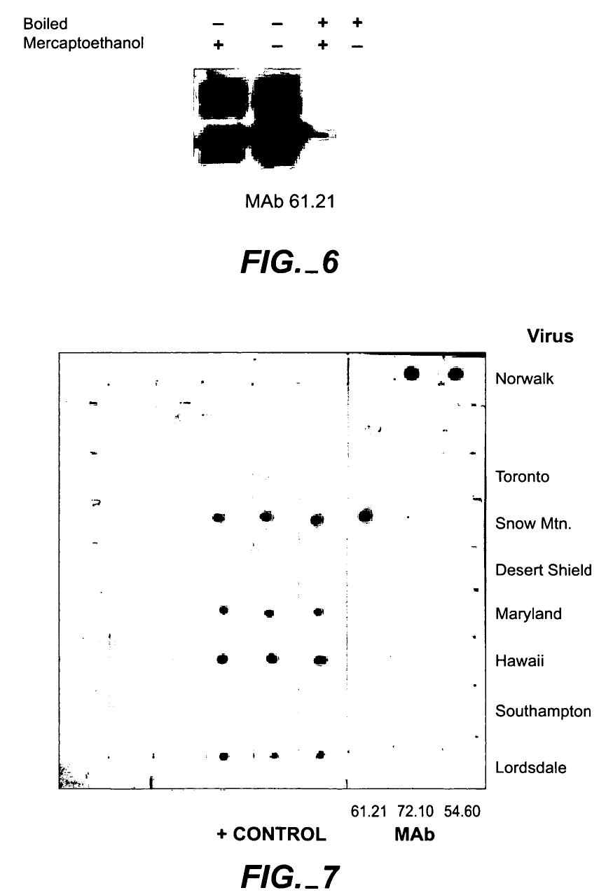

In Western blots, SMV61.21 only reacted with non-denatured protein (FIG. 6), which indicated that the antibody recognizes a conformation epitope. In dot blots, SMV61.21 only reacted with SMV and none of the other GI or GII viruses examined even though the PAP sequence and adjacent 1 to 2 amino acids are conserved among the viruses examined. The SMV amino acid three positions to the carboxy terminus from the PAP is a V, whereas the other GII viruses examined have a T are the corresponding position (FIG. 9). This difference in primary sequence may account for the reactivity of SMV61.21 only with SMV observed by dot blow (FIG. 7). The GI viruses have an F at the position corresponding to the SMV V and also have a deletion relative to the GII viruses (FIG. 9), which may result in their non-reactivity with SMV61.21.

### Example 3

### Blocking-Peptide ELISA Protocol

The wells of an Immulon 1 ELISA plate were coated with 10 ng/well of purified rNV by incubation overnight at 4° C. Plates were washed one with 0.05% Tween-20/PBS. To reduce nonspecific protein binding, each well is blocked with 3% bovine serum albumin (BSA)/PBS for 45 min. at room temperature. Reaction mixtures were prepared containing serial dilutions of peptides 1730, 1731, and 1732 beginning with 1:1 (1 mg/ml) and proceeding in 2-fold dilutions to 1:4 with 0.1 μg NV54.6 in a final reaction volume of 160 μl. Peptide IRR 1794 and diluent were run as negative controls. Antibody-peptide mixtures and controls were incubated for 45 min. at room temperature. BSA blocking solution was decanted from the plates which were washed three times with 0.05% Tween-20/PBS. Each well received 50 μl of antibody-peptide mixture or control and the plates were incubated for 1.5 hours at room temperature. Solutions were decanted from the plates which were washed three times with 0.05% Tween-20/PBS. Peroxidase-conjugated goat-antimouse IgG (secondary antibody) was diluted 1:3000 and 100 μl aliquots were added to each well. Plates were incubated for 1 hour at room temperature. During the last 10 min. of the incubation, OPD substrate was prepared by dissolving one 10 mg tablet in 10 ml of 0.05 M citrate buffer. Secondary antibody was decanted and the plates were washed three times with 0.05% Tween-20/PBS. 5 μl of 30% H₂O₂ was added to OPD substrate and 100 μl was immediately added to each well. Plates were incubated for 15 min. in the dark at room temperature. The reaction was stopped by the addition of 50 μl/well of 2.5 M sulfuric acid. Absorbance of each was well measured at 490 run on an ELISA plate reader.

The results shown in FIG. 4 demonstrate that peptides 1730, 1731, and 1732 substantially inhibit binding of NV54.6 to rNV in comparison to IRR 1794 and diluent. Repeating this study with SMV62.21 and rSMV shows that SMV62.21 inhibits bindings of rSMV to CaCo-2 cells.

### Example 4

### Peptide Blocking rNV or rSMV Binding to Host Cells

Peptide blocking of rNV and rSMV binding to CaCo-2 cells is performed similar to the procedure described above in which NV54.6 blocking of rNVs to CaCo-2 cells was demonstrated. Radiolabeled rNVs (15 μg/10⁵ cells) are mixed with serial dilutions of purified peptide 1730, 1731, 1731, 1800 or IRR 1794 as a negative control in 0.01 M PBS (final volume, 20 μl) for 1 h at 37° C. The serial dilutions of peptide are made with free peptide and peptide linked to a carrier, such as, BSA. The mixtures are chilled on ice, 10 μl 3% BSA in serum-free MEM is added to each reaction mixture and added to confluent monolayers in 96-well plates that are prewashed with cold serum-free MEM-1% BSA. Binding is assayed as described above. The results demonstrate that peptides 1730, 1731, 1732 reduce the binding of rNV and 1800 reduces binding of rSMV to CaCo-2 cells in a dose-dependent manner.

1. A recombinant peptide that inhibits binding of a Norovirus to a cell.

2. The peptide according to claim 1, wherein said peptide comprises a sequence corresponding to amino acids 133-137 of Norwalk virus.

3. The peptide according to claim 1, wherein the sequence of said peptide comprises WTRGX₉HX₁₀L (SEQ ID NO:95), wherein X₉ and X₁₀ can be independently any naturally occurring amino acid.

4. The peptide according to claim 1, wherein the sequence of said peptide is at least about 90% homologous to a peptide sequence selected from the group consisting of peptides 1730, 1731, and 1732.

5. The peptide according to claim 4, wherein said sequence is selected from the group consisting of peptides 1730, 1731, and 1732.

6. The peptide according to claim 1, wherein said Norovirus is a genogroup I Norovirus.

7. The peptide according to claim 1, wherein said Norovirus is a genogroup II Norovirus.

8. The peptide according to claim 1, wherein the sequence of said peptide corresponds to amino acids 319-327 of Snow Mountain virus.

9. The peptide according to claim 1, wherein the sequence of said peptide sequence comprises X₁₁—X₁₂—P-A-P—X₁₃—X₁₄—X₁₅—X₁₆ (SEQ ID NO:46), wherein X₁₁ is amino acid; X₁₂ is an amino acid having a linear or branched alkyl side chain; X₁₃ is an amino acid having a linear or branched alkyl side chain; X₁₄ is an amino acid having an acidic or hydrogen side chain; X₁₅ is an amino acid having a basic, alkyl, or hydroxyalkyl side chain; and X₁₆ is an amino acid having an aliphatic side chain or an imino acid.

10. The peptide according to claim 9, wherein said X₁₂ and X₁₃ are independently selected from the group consisting of leucine, isoleucine, valine, and alanine.

11. The peptide according to claim 10, wherein said sequence comprises WLPAPIDKL(SEQ ID NO:4).

12. The peptide according to claim 1, wherein said cell is a CaCo-2 cell.

13. The peptide according to claim 1, wherein said cell is an erythrocyte.

14. The peptide according to claim 1, wherein said peptide is formulated to be suitable for inducing an immune response in a subject.

15. An isolated antibody that binds to a Norovirus peptide epitope comprising an amino acid sequence corresponding to amino acids 133-137 of a Norwalk virus.

16. An isolated antibody that binds to a Norovirus peptide epitope comprising an amino acid sequence corresponding to amino acids 319-327 of Snow Mountain virus.

17. A method of inhibiting a Norovirus binding to a cell comprising contacting said cell with a peptide, whereby binding of said Norovirus to said cell is inhibited.

18. The method according to claim 17, wherein said peptide inhibits binding of the VP1 protein of said Norovirus to said cell.

19. The method according to claim 17, wherein the sequence of said peptide corresponds do to amino acids 133-137 or Norwalk virus.

20. The method according to claim 17, wherein the sequence of said peptide comprises WTRGX₉HX₁₀L (SEQ ID NO:95), wherein X₉ and X₁₀ can be independently any naturally occurring amino acid.

21. The method according to claim 17, wherein the sequence of said peptide is at least about 90% homologous to a peptide sequence selected from the group consisting of peptides 1730, 1731, and 1732.

22. The method according to claim 17, wherein the sequence of said peptide is selected from the group consisting of peptides 1730, 1731, and 1732.

23. The method according to claim 17, wherein said Norovirus is a Genogroup I Norovirus.

24. The method according to claim 17, wherein said Norovirus is a Genogroup II Norovirus.

25. The method according to claim 17, wherein the sequence of said peptide corresponds to amino acids 319-327 of Snow Mountain virus.

26. The method according to claim 17, wherein the sequence of said peptide comprises X₁₁—X₁₂—P-A-P—X₁₃—X₁₄—X₁₅—X₁₆ (SEQ ID NO:46), wherein X₁₁ is amino acid; X₁₂ is an amino acid having a linear or branched alkyl side chain; X₁₃ is an amino acid having a linear or branched alkyl side chain; X₁₄ is an amino acid having an acidic or hydrogen side chain; X₁₅ is an amino acid having a basic, alkyl, or hydroxyalkyl side chain; and X₁₆ is an amino acid having an aliphatic side chain or an imino acid.

27. The method according to claim 26, wherein said X₁₂ and X₁₃ are independently selected from the group consisting of leucine, isoleucine, valine, and alanine.

28. The method according to claim 27, wherein said sequence comprises WLPAPIDKL(SEQ ID NO:4).


SEQUENCE LISTING (159 sequences)

SEQ ID NO: 1 - protein, 8 aa, Artificial
  Synthetic
WTRGSHNL

SEQ ID NO: 2 - protein, 8 aa, Artificial
  Synthetic
WTRGGHGL

SEQ ID NO: 3 - protein, 8 aa, Artificial
  Synthetic
WTRGQHQL

SEQ ID NO: 4 - protein, 9 aa, Artificial
  Synthetic
WLPAPIDKL

SEQ ID NO: 5 - protein, 7 aa, Homo sapiens
RVYIHPF

SEQ ID NO: 6 - protein, 17 aa, Chiba virus
CVPPGFQSRTLSIAQAT

SEQ ID NO: 7 - protein, 17 aa, Desert Shield virus
CIPPGFAAQNISIAQAT

SEQ ID NO: 8 - protein, 17 aa, Grimsby virus
AVPPNFPAEGLSPSQVT

SEQ ID NO: 9 - protein, 17 aa, Hawaii virus
AIPPHFPLENLSPGQIT

SEQ ID NO: 10 - protein, 17 aa, Lordsdale virus
AVPPNFPTEGLSPSQVT

SEQ ID NO: 11 - protein, 17 aa, Maryland 145 virus
AVPPNFPTEGLSPSQVT

SEQ ID NO: 12 - protein, 17 aa, Mexico virus
AIPPNFPIDNLSAAQIT

SEQ ID NO: 13 - protein, 17 aa, Norwalk virus
CIPPGFGSHNLTIAQAT

SEQ ID NO: 14 - protein, 17 aa, Seto virus
CIPPGFGSHNLTIAQAT

SEQ ID NO: 15 - protein, 17 aa, Snow Mountain virus
AVPPHFPVENLSPQQIT

SEQ ID NO: 16 - protein, 17 aa, Southampton virus
CVPPGFTSSSLTIAQAT

SEQ ID NO: 17 - protein, 17 aa, Chiba virus
AFAAPAPAGFPDLGSCD

SEQ ID NO: 18 - protein, 17 aa, Desert Shield virus
AFESPAPIGFPDIGDCD

SEQ ID NO: 19 - protein, 18 aa, Grimsby virus
PTEEIPAPLGTPDFVGKI

SEQ ID NO: 20 - protein, 18 aa, Hawaii virus
PTEDVPAPLGTPDFLANI

SEQ ID NO: 21 - protein, 18 aa, Lordsdale virus
PTEEIPAPLGTPDFVGKI

SEQ ID NO: 22 - protein, 18 aa, Maryland 145 virus
PTEEIPAPLGTPDFVGKI

SEQ ID NO: 23 - protein, 18 aa, Mexico virus
PAEDIPAPLGTPDFRGKV

SEQ ID NO: 24 - protein, 17 aa, Norwalk virus
PFEGPAPIGFPDLGGCD

SEQ ID NO: 25 - protein, 17 aa, Seto virus
PFEGPAPIGFPDLGGCD

SEQ ID NO: 26 - protein, 18 aa, Snow Mountain virus
PSEDIPAPLGVPDFQGRV

SEQ ID NO: 27 - protein, 17 aa, Southampton virus
AFDSPAPVGFPDFGKCD

SEQ ID NO: 28 - protein, 530 aa, Norwalk virus
MMMASKDATSSVDGASGAGQLVPEVNASDPLAMDPVAGSSTAVATAGGVNPIDPWIINNFVQAPQGEFTISPNNTPGGVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLYPPTVEQKTRPFTLPNLPLSSLSNSRAPLPISGMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYIGVLSWVSPPSHPSGSQVDLWKIPNYGSSITEATHLAPSVYPPGFGEVLVFFMSKIPGPGAYSLPCLLPQEYISHLASEQAPTVGEAALLHYVDPDTGRTLGEFKAYPDGFLTCVPNGASSGPQQLPINGVFVFVSWVSRFYQLKPVGTASSARGRLGLRR

SEQ ID NO: 29 - protein, 530 aa, Norwalk virus; identical to SEQ ID NO: 28

SEQ ID NO: 30 - protein, 542 aa, Snow Mountain virus
MKMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVGVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTYESKTKPFTLPILTLGELSNSRFPVSIDQMYTSPNEVISVQCQNGRCTLDGELQGTTQLQVSGICAFKGEVTAHLGDNDHLYNITITNLNGSPFDPSEDIPAPLGVPDFQGRVFGVITQRDKGNAAGGSGPANRGHDAVVPTYTAGYTPKLGQVQIGTWQTDDLKVNGPVKFTPVGLNDTEHFNQWVVPRYAGALNLNTNLAPSVAPVFPGERLLFFRSYLPLKGGYGNPAIDCLLPGEWVQHFYQEAAPSMSEVALVRYINPDTGRALFEAKLHRAGFMTVSSNTSAPVVVPANGYFRFDSWVNQFYSLAPMGTGNGRRRIQ

SEQ ID NO: 31 - protein, 9 aa, Artificial
  Synthetic
WSLGQHRIS

SEQ ID NO: 32 - protein, 9 aa, Artificial
  Synthetic
WIRQGPFDK

SEQ ID NO: 33 - protein, 9 aa, Artificial
  Synthetic
WTRGMHQVS

SEQ ID NO: 34 - protein, 9 aa, Artificial
  Synthetic
WTRSEHNLA

SEQ ID NO: 35 - protein, 9 aa, Artificial
  Synthetic
WTLQWHTIQ

SEQ ID NO: 36 - protein, 9 aa, Artificial
  Synthetic
WSLDSHRLV

SEQ ID NO: 37 - protein, 9 aa, Artificial
  Synthetic
WTRGQHKLQ

SEQ ID NO: 38 - protein, 9 aa, Artificial
  Synthetic
WNIKQHSLY

SEQ ID NO: 39 - protein, 9 aa, Artificial
  Synthetic
WTRDQHQLH

SEQ ID NO: 40 - protein, 9 aa, Artificial
  Synthetic
WTLKNHTLS

SEQ ID NO: 41 - protein, 9 aa, Artificial
  Synthetic
WTRSMHSLL

SEQ ID NO: 42 - protein, 9 aa, Artificial
  Synthetic
WTRSMHSLV

SEQ ID NO: 43 - protein, 9 aa, Artificial
  Synthetic
WTRGDHQVW

SEQ ID NO: 44 - protein, 9 aa, Artificial
  Synthetic
WTRGDHQVX

SEQ ID NO: 45 - protein, 9 aa, Artificial
  Synthetic
WTRGMHQVW

SEQ ID NO: 46 - protein, 9 aa, Artificial
  Synthetic
XXPAPXXXX

SEQ ID NO: 47 - protein, 5 aa, Norwalk virus
  misc_feature (2)..(2): Xaa can be any naturally occurring amino acid
GXHXL

SEQ ID NO: 48 - protein, 9 aa, Artificial
  Synthetic
DIPAPLGVP

SEQ ID NO: 49 - protein, 9 aa, Artificial
  Synthetic
EIPAPLGTP

SEQ ID NO: 50 - protein, 9 aa, Artificial
  Synthetic
WIPAPIDKL

SEQ ID NO: 51 - protein, 9 aa, Artificial
  Synthetic
WVPAPLDKL

SEQ ID NO: 52 - protein, 9 aa, Artificial
  Synthetic
WIPAPLGKL

SEQ ID NO: 53 - protein, 9 aa, Artificial
  Synthetic
WVPAPLGKL

SEQ ID NO: 54 - protein, 9 aa, Artificial
  Synthetic
WIPAPLGVK

SEQ ID NO: 55 - protein, 9 aa, Artificial
  Synthetic
WIPAPLGTL

SEQ ID NO: 56 - protein, 9 aa, Artificial
  Synthetic
WVPAPLGTL

SEQ ID NO: 57 - protein, 9 aa, Artificial
  Synthetic
WIPAPLGVP

SEQ ID NO: 58 - protein, 9 aa, Artificial
  Synthetic
WIPAPLGTP

SEQ ID NO: 59 - protein, 9 aa, Artificial
  Synthetic
WVPAPLGTP

SEQ ID NO: 60 - protein, 8 aa, Artificial
  Synthetic
XPAPXGFP

SEQ ID NO: 61 - protein, 8 aa, Artificial
  Synthetic
GPAPIGFP

SEQ ID NO: 62 - protein, 8 aa, Artificial
  Synthetic
SPAPIGFP

SEQ ID NO: 63 - protein, 8 aa, Artificial
  Synthetic
SPAPVGFP

SEQ ID NO: 64 - protein, 8 aa, Artificial
  Synthetic
APAPAGFP

SEQ ID NO: 65 - protein, 9 aa, Artificial
  Synthetic
WLPAPIGFL

SEQ ID NO: 66 - protein, 9 aa, Artificial
  Synthetic
WLPAPIGFP

SEQ ID NO: 67 - protein, 8 aa, Artificial
  Synthetic
WPAPIDKL

SEQ ID NO: 68 - protein, 8 aa, Artificial
  Synthetic
WPAPIGKL

SEQ ID NO: 69 - protein, 8 aa, Artificial
  Synthetic
WPAPIGFL

SEQ ID NO: 70 - protein, 8 aa, Artificial
  Synthetic
WPAPIGFP

SEQ ID NO: 71 - protein, 9 aa, Artificial
  Synthetic
WLPAPVDKL

SEQ ID NO: 72 - protein, 9 aa, Artificial
  Synthetic
WLPAPVGKL

SEQ ID NO: 73 - protein, 9 aa, Artificial
  Synthetic
WLPAPVGFL

SEQ ID NO: 74 - protein, 9 aa, Artificial
  Synthetic
WLPAPVGFP

SEQ ID NO: 75 - protein, 8 aa, Artificial
  Synthetic
WPAPVDKL

SEQ ID NO: 76 - protein, 8 aa, Artificial
  Synthetic
WPAPVGKL

SEQ ID NO: 77 - protein, 8 aa, Artificial
  Synthetic
WPAPVGFL

SEQ ID NO: 78 - protein, 8 aa, Artificial
  Synthetic
WPAPVGFP

SEQ ID NO: 79 - protein, 9 aa, Artificial
  Synthetic
WLPAPADKL

SEQ ID NO: 80 - protein, 9 aa, Artificial
  Synthetic
WLPAPAGKL

SEQ ID NO: 81 - protein, 9 aa, Artificial
  Synthetic
WLPAPAGFL

SEQ ID NO: 82 - protein, 9 aa, Artificial
  Synthetic
WLPAPAGFP

SEQ ID NO: 83 - protein, 8 aa, Artificial
  Synthetic
WPAPADKL

SEQ ID NO: 84 - protein, 8 aa, Artificial
  Synthetic
WPAPAGKL

SEQ ID NO: 85 - protein, 8 aa, Artificial
  Synthetic
WPAPAGFL

SEQ ID NO: 86 - protein, 8 aa, Artificial
  Synthetic
WPAPAGFP

SEQ ID NO: 87 - protein, 5 aa, Artificial
  Synthetic
GSHNL

SEQ ID NO: 88 - protein, 8 aa, Artificial
  Synthetic
WTRAAQNI

SEQ ID NO: 89 - protein, 8 aa, Artificial
  Synthetic
WTRTSSSL

SEQ ID NO: 90 - protein, 8 aa, Artificial
  Synthetic
WTRQSRTL

SEQ ID NO: 91 - protein, 8 aa, Artificial
  Synthetic
WTRPVENL

SEQ ID NO: 92 - protein, 8 aa, Artificial
  Synthetic
WTRPLENL

SEQ ID NO: 93 - protein, 8 aa, Artificial
  Synthetic
WTRPTEGL

SEQ ID NO: 94 - DNA, 24 bp, Artificial
  Synthetic
tggactcgtggttctcataatctt

SEQ ID NO: 95 - protein, 8 aa, Artificial
  Synthetic
WTRGXHXL

SEQ ID NO: 96 - protein, 8 aa, Artificial
  Synthetic
WTRGXHXL

SEQ ID NO: 97 - protein, 9 aa, Artificial
  Synthetic
WXXXXXXXX

SEQ ID NO: 98 - protein, 8 aa, Artificial
  Synthetic
WXXXXXXX

SEQ ID NO: 99 - protein, 9 aa, Artificial
  Synthetic
WIRQGPFDK

SEQ ID NO: 100 - protein, 9 aa, Artificial
  Synthetic
WLPAPLDKL

SEQ ID NO: 101 - protein, 9 aa, Artificial
  Synthetic
WIPAPLGVL

SEQ ID NO: 102 - protein, 9 aa, Artificial
  Synthetic
WIPAPLGVL

SEQ ID NO: 103 - protein, 9 aa, Artificial
  Synthetic
DIPAPLGTP

SEQ ID NO: 104 - protein, 9 aa, Artificial
  Synthetic
DVPAPLGTP

SEQ ID NO: 105 - protein, 9 aa, Artificial
  Synthetic
WLPAPIGKL

SEQ ID NO: 106 - protein, 8 aa, Artificial
  Synthetic
WTRPAEGL

SEQ ID NO: 107 - protein, 8 aa, Artificial
  Synthetic
WLSPTEGL

SEQ ID NO: 108 - protein, 8 aa, Artificial
  Synthetic
WLSGSHNL

SEQ ID NO: 109 - protein, 8 aa, Artificial
  Synthetic
WIRGSHNL

SEQ ID NO: 110 - protein, 8 aa, Artificial
  Synthetic
WNIGSHNL

SEQ ID NO: 111 - protein, 8 aa, Artificial
  Synthetic
WLSAAQNI

SEQ ID NO: 112 - protein, 8 aa, Artificial
  Synthetic
WIRAAQNI

SEQ ID NO: 113 - protein, 8 aa, Artificial
  Synthetic
WNIAAQNI

SEQ ID NO: 114 - protein, 8 aa, Artificial
  Synthetic
WLSTSSSL

SEQ ID NO: 115 - protein, 8 aa, Artificial
  Synthetic
WIRTSSSL

SEQ ID NO: 116 - protein, 8 aa, Artificial
  Synthetic
WNITSSSL

SEQ ID NO: 117 - protein, 8 aa, Artificial
  Synthetic
WLSQSTRL

SEQ ID NO: 118 - protein, 8 aa, Artificial
  Synthetic
WIRQSTRL

SEQ ID NO: 119 - protein, 8 aa, Artificial
  Synthetic
WNIQSTRL

SEQ ID NO: 120 - protein, 8 aa, Artificial
  Synthetic
WLSPVENL

SEQ ID NO: 121 - protein, 8 aa, Artificial
  Synthetic
WIRPVENL

SEQ ID NO: 122 - protein, 8 aa, Artificial
  Synthetic
WNIPVENL

SEQ ID NO: 123 - protein, 8 aa, Artificial
  Synthetic
WLSPLENL

SEQ ID NO: 124 - protein, 8 aa, Artificial
  Synthetic
WIRPLENL

SEQ ID NO: 125 - protein, 8 aa, Artificial
  Synthetic
WNIPLENL

SEQ ID NO: 126 - protein, 8 aa, Artificial
  Synthetic
WIRPTEGL

SEQ ID NO: 127 - protein, 8 aa, Artificial
  Synthetic
WNIPTEGL

SEQ ID NO: 128 - protein, 8 aa, Artificial
  Synthetic
WLSPAEGL

SEQ ID NO: 129 - protein, 8 aa, Artificial
  Synthetic
WIRPAEGL

SEQ ID NO: 130 - protein, 8 aa, Artificial
  Synthetic
WNIPAEGL

SEQ ID NO: 131 - protein, 8 aa, Artificial
  Synthetic
WTRPIDNL

SEQ ID NO: 132 - protein, 8 aa, Artificial
  Synthetic
WLSPIDNL

SEQ ID NO: 133 - protein, 8 aa, Artificial
  Synthetic
WIRPIDNL

SEQ ID NO: 134 - protein, 8 aa, Artificial
  Synthetic
WNIPIDNL

SEQ ID NO: 135 - protein, 8 aa, Artificial
  Synthetic
WLSQSRTL

SEQ ID NO: 136 - protein, 8 aa, Artificial
  Synthetic
WIRQSRTL

SEQ ID NO: 137 - protein, 8 aa, Artificial
  Synthetic
WNIQSRTL

SEQ ID NO: 138 - protein, 8 aa, Artificial
  Synthetic
WTRPVENI

SEQ ID NO: 139 - protein, 8 aa, Artificial
  Synthetic
WLSPVENI

SEQ ID NO: 140 - protein, 8 aa, Artificial
  Synthetic
WIRPVENI

SEQ ID NO: 141 - protein, 8 aa, Artificial
  Synthetic
WNIPVENI

SEQ ID NO: 142 - protein, 9 aa, Artificial
  Synthetic
XXXXXXXXX

SEQ ID NO: 143 - protein, 9 aa, Artificial
  Synthetic
WTRGXHXLX

SEQ ID NO: 144 - protein, 9 aa, Artificial
  Synthetic
WTXXXXXLX

SEQ ID NO: 145 - protein, 9 aa, Artificial
  Synthetic
XXPAPXXXX

SEQ ID NO: 146 - protein, 5 aa, Snow Mountain virus
IPAPL

SEQ ID NO: 147 - protein, 9 aa, Artificial
  Synthetic
WTRGMHQVS

SEQ ID NO: 148 - protein, 9 aa, Artificial
  Synthetic
WTRSEHNLA

SEQ ID NO: 149 - protein, 9 aa, Artificial
  Synthetic
WTLQWHTIQ

SEQ ID NO: 150 - protein, 9 aa, Artificial
  Synthetic
WSLDSHRLV

SEQ ID NO: 151 - protein, 9 aa, Artificial
  Synthetic
WTRGQHKLQ

SEQ ID NO: 152 - protein, 9 aa, Artificial
  Synthetic
WNIKQHSLY

SEQ ID NO: 153 - protein, 9 aa, Artificial
  Synthetic
WTRDQHQLH

SEQ ID NO: 154 - protein, 9 aa, Artificial
  Synthetic
WTLKNHTLS

SEQ ID NO: 155 - protein, 9 aa, Artificial
  Synthetic
WTRSMHSLL

SEQ ID NO: 156 - protein, 9 aa, Artificial
  Synthetic
WTRSMHSLV

SEQ ID NO: 157 - protein, 9 aa, Artificial
  Synthetic
WTRGDHQVW

SEQ ID NO: 158 - protein, 9 aa, Artificial
  Synthetic
WTRGDHQVX

SEQ ID NO: 159 - protein, 9 aa, Artificial
  Synthetic
WTRGMHQVW